SEQUENCE LISTING (11 sequences)

SEQ ID NO: 1 - DNA, 2016 bp, Pichia Pastoris
atgcagagtaattcggagagagactcttcgcctagtgactcaaatagcaccattgagttgcaaagatccgagaacgaatatgatcacctaactaatacgataatggaagatctggggcaaaaacttaaccactacaaggaatcccaggacacgagctccagccatattttacacttacctactgaggttttgctactcattttatcatttgtgacttcgaagactgatcttcttagttttatgttgacatgtagaaagttcggagacctggttagcggtttgctctggttcagacctggtatttccaatgcatacgtctataaagaaatgatcagaataatgagaatacctccagagaagacattttgggactacaaaaagtttatcagaagattgaatctgtccctggtttctaacttggttgaggatgagttcctatatgcattcagtggttgccccaacctggaaaggatcacattagtgaattgcagtaaagttactgctgattctgtggcgacaatattgaaggatgcatccaaccttcagtctattgaccttacaggagttgtgaatatcacagatggagtctactacagtttagcacgccactgtaagaaactgcagggtctatatgccccaggttctatggctgtttccaagaacgcagtgtacactctcatatccaattgcccaatgctgaaaagaatcaaactgagtgaatgtgtgggagtagacgatgagattgttgtgaaattggtgagagaatgtaaaaatctcgtcgaattagaccttcatgggtgtatcagagttaccgattatgctctagttgtgctttttgaagaattggaatatttgagagagttcaaaatctcaatgaatgatcatataacagagagatgcttccttgggctaccaaacgagccctacttggataagcttaggataattgatttcactagttgcagcaatgttaacgacaaacttgtcatcaagttagttcaattagcacccaagttgaggcatattgtattgtctaagtgtaccaaaataacggactcgtccttgagagccttagcaactttgggcaagtgcttgcactacttgcatctgggacattgtattaacataacagattttggagtctgtcatctgcttagaaattgtcatcgacttcagtatgtcgatcttgcatgctgtcaagagctgaccaatgacaccttgtttgaactatctcagttaccaagattgagaagaattggcttggtgaaatgtcacaatataaccgatcatggcattttgtatctagcaaataaccggagatcgccagacgatactttagaaagagtacatttatcatattgtacacagattagcatatttcctatctacaagttactaatggcgtgtcgcagactgacacacctatcattaacaggtatcagagacttcttgagaagtgatattacaagattttgccgagatcctcccaatgactttactcaatctcaaagagatatgttttgtgttttcagtggtgacggggtccggaagcttcgagatcacctttctagtctctaccaccaacagcaacaaattaacagatacgttaactctcaaaatataggaaacttaagggatgacggagagactttgaatgagatcttccagtatattgcaaatccagccacccctggacaactacccccaagggtccaagaactcgttgaagcaagacggagaaacagaaatcaagaacgcataatgactaacactgttaactttcctgaacaaattagaaggttgtctctcctgcctccggaacaacaacaggctttgccacaaccaattcgtcaactgattgcccaagccactgcatctccgctatcttttcctttacaagatcaggagccacaacagcagcaacaacaagaaaggggtcttggcattccgccagttgataacttcagtccggtagttgatgaagggcaagaatatgatgaagaccaagagatggaa

SEQ ID NO: 2 - DNA, 2016 bp, Artificial Sequence
  PpGRR1 (L410P) mutant ORF
atgcagagtaattcggagagagactcttcgcctagtgactcaaatagcaccattgagttgcaaagatccgagaacgaatatgatcacctaactaatacgataatggaagatctggggcaaaaacttaaccactacaaggaatcccaggacacgagctccagccatattttacacttacctactgaggttttgctactcattttatcatttgtgacttcgaagactgatcttcttagttttatgttgacatgtagaaagttcggagacctggttagcggtttgctctggttcagacctggtatttccaatgcatacgtctataaagaaatgatcagaataatgagaatacctccagagaagacattttgggactacaaaaagtttatcagaagattgaatctgtccctggtttctaacttggttgaggatgagttcctatatgcattcagtggttgccccaacctggaaaggatcacattagtgaattgcagtaaagttactgctgattctgtggcgacaatattgaaggatgcatccaaccttcagtctattgaccttacaggagttgtgaatatcacagatggagtctactacagtttagcacgccactgtaagaaactgcagggtctatatgccccaggttctatggctgtttccaagaacgcagtgtacactctcatatccaattgcccaatgctgaaaagaatcaaactgagtgaatgtgtgggagtagacgatgagattgttgtgaaattggtgagagaatgtaaaaatctcgtcgaattagaccttcatgggtgtatcagagttaccgattatgctctagttgtgctttttgaagaattggaatatttgagagagttcaaaatctcaatgaatgatcatataacagagagatgcttccttgggctaccaaacgagccctacttggataagcttaggataattgatttcactagttgcagcaatgttaacgacaaacttgtcatcaagttagttcaattagcacccaagttgaggcatattgtattgtctaagtgtaccaaaataacggactcgtccttgagagccttagcaactttgggcaagtgcttgcactacttgcatctgggacattgtattaacataacagattttggagtctgtcatctgcttagaaattgtcatcgacttcagtatgtcgatcttgcatgctgtcaagagctgaccaatgacaccttgtttgaacaatctcagttaccaagattgagaagaattggcttggtgaaatgtcacaatataaccgatcatggcattttgtatctagcaaataaccggagatcgccagacgatactttagaaagagtacatttatcatattgtacacagattagcatatttcctatctacaagttactaatggcgtgtcgcagactgacacacctatcattaacaggtatcagagacttcttgagaagtgatattacaagattttgccgagatcctcccaatgactttactcaatctcaaagagatatgttttgtgttttcagtggtgacggggtccggaagcttcgagatcacctttctagtctctaccaccaacagcaacaaattaacagatacgttaactctcaaaatataggaaacttaagggatgacggagagactttgaatgagatcttccagtatattgcaaatccagccacccctggacaactacccccaagggtccaagaactcgttgaagcaagacggagaaacagaaatcaagaacgcataatgactaacactgttaactttcctgaacaaattagaaggttgtctctcctgcctccggaacaacaacaggctttgccacaaccaattcgtcaactgattgcccaagccactgcatctccgctatcttttcctttacaagatcaggagccacaacagcagcaacaacaagaaaggggtcttggcattccgccagttgataacttcagtccggtagttgatgaagggcaagaatatgatgaagaccaagagatggaa

SEQ ID NO: 3 - DNA, 2016 bp, Artificial Sequence
  PpGRR1 (L451S) mutant ORF
atgcagagtaattcggagagagactcttcgcctagtgactcaaatagcaccattgagttgcaaagatccgagaacgaatatgatcacctaactaatacgataatggaagatctggggcaaaaacttaaccactacaaggaatcccaggacacgagctccagccatattttacacttacctactgaggttttgctactcattttatcatttgtgacttcgaagactgatcttcttagttttatgttgacatgtagaaagttcggagacctggttagcggtttgctctggttcagacctggtatttccaatgcatacgtctataaagaaatgatcagaataatgagaatacctccagagaagacattttgggactacaaaaagtttatcagaagattgaatctgtccctggtttctaacttggttgaggatgagttcctatatgcattcagtggttgccccaacctggaaaggatcacattagtgaattgcagtaaagttactgctgattctgtggcgacaatattgaaggatgcatccaaccttcagtctattgaccttacaggagttgtgaatatcacagatggagtctactacagtttagcacgccactgtaagaaactgcagggtctatatgccccaggttctatggctgtttccaagaacgcagtgtacactctcatatccaattgcccaatgctgaaaagaatcaaactgagtgaatgtgtgggagtagacgatgagattgttgtgaaattggtgagagaatgtaaaaatctcgtcgaattagaccttcatgggtgtatcagagttaccgattatgctctagttgtgctttttgaagaattggaatatttgagagagttcaaaatctcaatgaatgatcatataacagagagatgcttccttgggctaccaaacgagccctacttggataagcttaggataattgatttcactagttgcagcaatgttaacgacaaacttgtcatcaagttagttcaattagcacccaagttgaggcatattgtattgtctaagtgtaccaaaataacggactcgtccttgagagccttagcaactttgggcaagtgcttgcactacttgcatctgggacattgtattaacataacagattttggagtctgtcatctgcttagaaattgtcatcgacttcagtatgtcgatcttgcatgctgtcaagagctgaccaatgacaccttgtttgaactatctcagttaccaagattgagaagaattggcttggtgaaatgtcacaatataaccgatcatggcattttgtatctagcaaataaccggagatcgccagacgatactttagaaagagtacattcatcatattgtacacagattagcatatttcctatctacaagttactaatggcgtgtcgcagactgacacacctatcattaacaggtatcagagacttcttgagaagtgatattacaagattttgccgagatcctcccaatgactttactcaatctcaaagagatatgttttgtgttttcagtggtgacggggtccggaagcttcgagatcacctttctagtctctaccaccaacagcaacaaattaacagatacgttaactctcaaaatataggaaacttaagggatgacggagagactttgaatgagatcttccagtatattgcaaatccagccacccctggacaactacccccaagggtccaagaactcgttgaagcaagacggagaaacagaaatcaagaacgcataatgactaacactgttaactttcctgaacaaattagaaggttgtctctcctgcctccggaacaacaacaggctttgccacaaccaattcgtcaactgattgcccaagccactgcatctccgctatcttttcctttacaagatcaggagccacaacagcagcaacaacaagaaaggggtcttggcattccgccagttgataacttcagtccggtagttgatgaagggcaagaatatgatgaagaccaagagatggaa

SEQ ID NO: 4 - DNA, 2016 bp, Artificial Sequence
  PpGRR1 (S452L) mutant ORF
atgcagagtaattcggagagagactcttcgcctagtgactcaaatagcaccattgagttgcaaagatccgagaacgaatatgatcacctaactaatacgataatggaagatctggggcaaaaacttaaccactacaaggaatcccaggacacgagctccagccatattttacacttacctactgaggttttgctactcattttatcatttgtgacttcgaagactgatcttcttagttttatgttgacatgtagaaagttcggagacctggttagcggtttgctctggttcagacctggtatttccaatgcatacgtctataaagaaatgatcagaataatgagaatacctccagagaagacattttgggactacaaaaagtttatcagaagattgaatctgtccctggtttctaacttggttgaggatgagttcctatatgcattcagtggttgccccaacctggaaaggatcacattagtgaattgcagtaaagttactgctgattctgtggcgacaatattgaaggatgcatccaaccttcagtctattgaccttacaggagttgtgaatatcacagatggagtctactacagtttagcacgccactgtaagaaactgcagggtctatatgccccaggttctatggctgtttccaagaacgcagtgtacactctcatatccaattgcccaatgctgaaaagaatcaaactgagtgaatgtgtgggagtagacgatgagattgttgtgaaattggtgagagaatgtaaaaatctcgtcgaattagaccttcatgggtgtatcagagttaccgattatgctctagttgtgctttttgaagaattggaatatttgagagagttcaaaatctcaatgaatgatcatataacagagagatgcttccttgggctaccaaacgagccctacttggataagcttaggataattgatttcactagttgcagcaatgttaacgacaaacttgtcatcaagttagttcaattagcacccaagttgaggcatattgtattgtctaagtgtaccaaaataacggactcgtccttgagagccttagcaactttgggcaagtgcttgcactacttgcatctgggacattgtattaacataacagattttggagtctgtcatctgcttagaaattgtcatcgacttcagtatgtcgatcttgcatgctgtcaagagctgaccaatgacaccttgtttgaactatctcagttaccaagattgagaagaattggcttggtgaaatgtcacaatataaccgatcatggcattttgtatctagcaaataaccggagatcgccagacgatactttagaaagagtacatttattatattgtacacagattagcatatttcctatctacaagttactaatggcgtgtcgcagactgacacacctatcattaacaggtatcagagacttcttgagaagtgatattacaagattttgccgagatcctcccaatgactttactcaatctcaaagagatatgttttgtgttttcagtggtgacggggtccggaagcttcgagatcacctttctagtctctaccaccaacagcaacaaattaacagatacgttaactctcaaaatataggaaacttaagggatgacggagagactttgaatgagatcttccagtatattgcaaatccagccacccctggacaactacccccaagggtccaagaactcgttgaagcaagacggagaaacagaaatcaagaacgcataatgactaacactgttaactttcctgaacaaattagaaggttgtctctcctgcctccggaacaacaacaggctttgccacaaccaattcgtcaactgattgcccaagccactgcatctccgctatcttttcctttacaagatcaggagccacaacagcagcaacaacaagaaaggggtcttggcattccgccagttgataacttcagtccggtagttgatgaagggcaagaatatgatgaagaccaagagatggaa

SEQ ID NO: 5 - DNA, 2016 bp, Artificial Sequence
  PpGRR1 (R617C) mutant ORF
atgcagagtaattcggagagagactcttcgcctagtgactcaaatagcaccattgagttgcaaagatccgagaacgaatatgatcacctaactaatacgataatggaagatctggggcaaaaacttaaccactacaaggaatcccaggacacgagctccagccatattttacacttacctactgaggttttgctactcattttatcatttgtgacttcgaagactgatcttcttagttttatgttgacatgtagaaagttcggagacctggttagcggtttgctctggttcagacctggtatttccaatgcatacgtctataaagaaatgatcagaataatgagaatacctccagagaagacattttgggactacaaaaagtttatcagaagattgaatctgtccctggtttctaacttggttgaggatgagttcctatatgcattcagtggttgccccaacctggaaaggatcacattagtgaattgcagtaaagttactgctgattctgtggcgacaatattgaaggatgcatccaaccttcagtctattgaccttacaggagttgtgaatatcacagatggagtctactacagtttagcacgccactgtaagaaactgcagggtctatatgccccaggttctatggctgtttccaagaacgcagtgtacactctcatatccaattgcccaatgctgaaaagaatcaaactgagtgaatgtgtgggagtagacgatgagattgttgtgaaattggtgagagaatgtaaaaatctcgtcgaattagaccttcatgggtgtatcagagttaccgattatgctctagttgtgctttttgaagaattggaatatttgagagagttcaaaatctcaatgaatgatcatataacagagagatgcttccttgggctaccaaacgagccctacttggataagcttaggataattgatttcactagttgcagcaatgttaacgacaaacttgtcatcaagttagttcaattagcacccaagttgaggcatattgtattgtctaagtgtaccaaaataacggactcgtccttgagagccttagcaactttgggcaagtgcttgcactacttgcatctgggacattgtattaacataacagattttggagtctgtcatctgcttagaaattgtcatcgacttcagtatgtcgatcttgcatgctgtcaagagctgaccaatgacaccttgtttgaactatctcagttaccaagattgagaagaattggcttggtgaaatgtcacaatataaccgatcatggcattttgtatctagcaaataaccggagatcgccagacgatactttagaaagagtacatttatcatattgtacacagattagcatatttcctatctacaagttactaatggcgtgtcgcagactgacacacctatcattaacaggtatcagagacttcttgagaagtgatattacaagattttgccgagatcctcccaatgactttactcaatctcaaagagatatgttttgtgttttcagtggtgacggggtccggaagcttcgagatcacctttctagtctctaccaccaacagcaacaaattaacagatacgttaactctcaaaatataggaaacttaagggatgacggagagactttgaatgagatcttccagtatattgcaaatccagccacccctggacaactacccccaagggtccaagaactcgttgaagcaagacggagaaacagaaatcaagaacgcataatgactaacactgttaactttcctgaacaaattagaaggttgtctctcctgcctccggaacaacaacaggctttgccacaaccaatttgtcaactgattgcccaagccactgcatctccgctatcttttcctttacaagatcaggagccacaacagcagcaacaacaagaaaggggtcttggcattccgccagttgataacttcagtccggtagttgatgaagggcaagaatatgatgaagaccaagagatggaa

SEQ ID NO: 6 - protein, 672 aa, Pichia Pastoris
MQSNSERDSSPSDSNSTIELQRSENEYDHLTNTIMEDLGQKLNHYKESQDTSSSHILHLPTEVLLLILSFVTSKTDLLSFMLTCRKFGDLVSGLLWFRPGISNAYVYKEMIRIMRIPPEKTFWDYKKFIRRLNLSLVSNLVEDEFLYAFSGCPNLERITLVNCSKVTADSVATILKDASNLQSIDLTGVVNITDGVYYSLARHCKKLQGLYAPGSMAVSKNAVYTLISNCPMLKRIKLSECVGVDDEIVVKLVRECKNLVELDLHGCIRVTDYALVVLFEELEYLREFKISMNDHITERCFLGLPNEPYLDKLRIIDFTSCSNVNDKLVIKLVQLAPKLRHIVLSKCTKITDSSLRALATLGKCLHYLHLGHCINITDFGVCHLLRNCHRLQYVDLACCQELTNDTLFELSQLPRLRRIGLVKCHNITDHGILYLANNRRSPDDTLERVHLSYCTQISIFPIYKLLMACRRLTHLSLTGIRDFLRSDITRFCRDPPNDFTQSQRDMFCVFSGDGVRKLRDHLSSLYHQQQQINRYVNSQNIGNLRDDGETLNEIFQYIANPATPGQLPPRVQELVEARRRNRNQERIMTNTVNFPEQIRRLSLLPPEQQQALPQPIRQLIAQATASPLSFPLQDQEPQQQQQQERGLGIPPVDNFSPVVDEGQEYDEDQEME

SEQ ID NO: 7 - protein, 672 aa, Artificial Sequence
  PpGRR1 (L410P) mutant
MQSNSERDSSPSDSNSTIELQRSENEYDHLTNTIMEDLGQKLNHYKESQDTSSSHILHLPTEVLLLILSFVTSKTDLLSFMLTCRKFGDLVSGLLWFRPGISNAYVYKEMIRIMRIPPEKTFWDYKKFIRRLNLSLVSNLVEDEFLYAFSGCPNLERITLVNCSKVTADSVATILKDASNLQSIDLTGVVNITDGVYYSLARHCKKLQGLYAPGSMAVSKNAVYTLISNCPMLKRIKLSECVGVDDEIVVKLVRECKNLVELDLHGCIRVTDYALVVLFEELEYLREFKISMNDHITERCFLGLPNEPYLDKLRIIDFTSCSNVNDKLVIKLVQLAPKLRHIVLSKCTKITDSSLRALATLGKCLHYLHLGHCINITDFGVCHLLRNCHRLQYVDLACCQELTNDTLFEQSQLPRLRRIGLVKCHNITDHGILYLANNRRSPDDTLERVHLSYCTQISIFPIYKLLMACRRLTHLSLTGIRDFLRSDITRFCRDPPNDFTQSQRDMFCVFSGDGVRKLRDHLSSLYHQQQQINRYVNSQNIGNLRDDGETLNEIFQYIANPATPGQLPPRVQELVEARRRNRNQERIMTNTVNFPEQIRRLSLLPPEQQQALPQPIRQLIAQATASPLSFPLQDQEPQQQQQQERGLGIPPVDNFSPVVDEGQEYDEDQEME

SEQ ID NO: 8 - protein, 672 aa, Artificial Sequence
  PpGRR1 (L451S) mutant
MQSNSERDSSPSDSNSTIELQRSENEYDHLTNTIMEDLGQKLNHYKESQDTSSSHILHLPTEVLLLILSFVTSKTDLLSFMLTCRKFGDLVSGLLWFRPGISNAYVYKEMIRIMRIPPEKTFWDYKKFIRRLNLSLVSNLVEDEFLYAFSGCPNLERITLVNCSKVTADSVATILKDASNLQSIDLTGVVNITDGVYYSLARHCKKLQGLYAPGSMAVSKNAVYTLISNCPMLKRIKLSECVGVDDEIVVKLVRECKNLVELDLHGCIRVTDYALVVLFEELEYLREFKISMNDHITERCFLGLPNEPYLDKLRIIDFTSCSNVNDKLVIKLVQLAPKLRHIVLSKCTKITDSSLRALATLGKCLHYLHLGHCINITDFGVCHLLRNCHRLQYVDLACCQELTNDTLFELSQLPRLRRIGLVKCHNITDHGILYLANNRRSPDDTLERVHSSYCTQISIFPIYKLLMACRRLTHLSLTGIRDFLRSDITRFCRDPPNDFTQSQRDMFCVFSGDGVRKLRDHLSSLYHQQQQINRYVNSQNIGNLRDDGETLNEIFQYIANPATPGQLPPRVQELVEARRRNRNQERIMTNTVNFPEQIRRLSLLPPEQQQALPQPIRQLIAQATASPLSFPLQDQEPQQQQQQERGLGIPPVDNFSPVVDEGQEYDEDQEME

SEQ ID NO: 9 - protein, 672 aa, Artificial Sequence
  PpGRR1 (S452L) mutant
MQSNSERDSSPSDSNSTIELQRSENEYDHLTNTIMEDLGQKLNHYKESQDTSSSHILHLPTEVLLLILSFVTSKTDLLSFMLTCRKFGDLVSGLLWFRPGISNAYVYKEMIRIMRIPPEKTFWDYKKFIRRLNLSLVSNLVEDEFLYAFSGCPNLERITLVNCSKVTADSVATILKDASNLQSIDLTGVVNITDGVYYSLARHCKKLQGLYAPGSMAVSKNAVYTLISNCPMLKRIKLSECVGVDDEIVVKLVRECKNLVELDLHGCIRVTDYALVVLFEELEYLREFKISMNDHITERCFLGLPNEPYLDKLRIIDFTSCSNVNDKLVIKLVQLAPKLRHIVLSKCTKITDSSLRALATLGKCLHYLHLGHCINITDFGVCHLLRNCHRLQYVDLACCQELTNDTLFELSQLPRLRRIGLVKCHNITDHGILYLANNRRSPDDTLERVHLLYCTQISIFPIYKLLMACRRLTHLSLTGIRDFLRSDITRFCRDPPNDFTQSQRDMFCVFSGDGVRKLRDHLSSLYHQQQQINRYVNSQNIGNLRDDGETLNEIFQYIANPATPGQLPPRVQELVEARRRNRNQERIMTNTVNFPEQIRRLSLLPPEQQQALPQPIRQLIAQATASPLSFPLQDQEPQQQQQQERGLGIPPVDNFSPVVDEGQEYDEDQEME

SEQ ID NO: 10 - protein, 672 aa, Artificial Sequence
  PpGRR1 (R617C) mutant
MQSNSERDSSPSDSNSTIELQRSENEYDHLTNTIMEDLGQKLNHYKESQDTSSSHILHLPTEVLLLILSFVTSKTDLLSFMLTCRKFGDLVSGLLWFRPGISNAYVYKEMIRIMRIPPEKTFWDYKKFIRRLNLSLVSNLVEDEFLYAFSGCPNLERITLVNCSKVTADSVATILKDASNLQSIDLTGVVNITDGVYYSLARHCKKLQGLYAPGSMAVSKNAVYTLISNCPMLKRIKLSECVGVDDEIVVKLVRECKNLVELDLHGCIRVTDYALVVLFEELEYLREFKISMNDHITERCFLGLPNEPYLDKLRIIDFTSCSNVNDKLVIKLVQLAPKLRHIVLSKCTKITDSSLRALATLGKCLHYLHLGHCINITDFGVCHLLRNCHRLQYVDLACCQELTNDTLFELSQLPRLRRIGLVKCHNITDHGILYLANNRRSPDDTLERVHLSYCTQISIFPIYKLLMACRRLTHLSLTGIRDFLRSDITRFCRDPPNDFTQSQRDMFCVFSGDGVRKLRDHLSSLYHQQQQINRYVNSQNIGNLRDDGETLNEIFQYIANPATPGQLPPRVQELVEARRRNRNQERIMTNTVNFPEQIRRLSLLPPEQQQALPQPICQLIAQATASPLSFPLQDQEPQQQQQQERGLGIPPVDNFSPVVDEGQEYDEDQEME

SEQ ID NO: 11 - protein, 1151 aa, Sachromyces cerevisiae
MDQDNNNHNDSNRLHPPDIHPNLGPQLWLNSSGDFDDNNNNNNNNNNNNSTRPQMPSRTRETATSERNASEVRDATLNNIFRFDSIQRETLLPTNNGQPLNQNFSLTFQPQQQTNALNGIDINTVNTNLMNGVNVQIDQLNRLLPNLPEEERKQIHEFKLIVGKKIQEFLVVIEKRRKKILNEIELDNLKLKELRIDNSPQAISYLHKLQRMRLRALETENMEIRNLRLKILTIIEEYKKSLYAYCHSKLRGQQVENPTDNFIIWINSIDTTESSDLKEGLQDLSRYSRQFINNVLSNPSNQNICTSVTRRSPVFALNMLPSEILHLILDKLNQKYDIVKFLTVSKLWAEIIVKILYYRPHINKKSQLDLFLRTMKLTSEETVFNYRLMIKRLNFSFVGDYMHDTELNYFVGCKNLERLTLVFCKHITSVPISAVLRGCKFLQSVDITGIRDVSDDVFDTLATYCPRVQGFYVPQARNVTFDSLRNFIVHSPMLKRIKITANNNMNDELVELLANKCPLLVEVDITLSPNVTDSSLLKLLTRLVQLREFRITHNTNITDNLFQELSKVVDDMPSLRLIDLSGCENITDKTIESIVNLAPKLRNVFLGKCSRITDASLFQLSKLGKNLQTVHFGHCFNITDNGVRALFHSCTRIQYVDFACCTNLTNRTLYELADLPKLKRIGLVKCTQMTDEGLLNMVSLRGRNDTLERVHLSYCSNLTIYPIYELLMSCPRLSHLSLTAVPSFLRPDITMYCRPAPSDFSENQRQIFCVFSGKGVHKLRHYLVNLTSPAFGPHVDVNDVLTKYIRSKNLIFNGETLEDALRRIITDLNQDSAAIIAATGLNQINGLNNDFLFQNINFERIDEVFSWYLNTFDGIRMSSEEVNSLLLQVNKTFCEDPFSDVDDDQDYVVAPGVNREINSEMCHIVRKFHELNDHIDDFEVNVASLVRVQFQFTGFLLHEMTQTYMQMIELNRQICLVQKTVQESGNIDYQKGLLIWRLLFIDKFIMVVQKYKLSTVVLRLYLKDNITLLTRQRELLIAHQRSAWNNNNDNDANRNANNIVNIVSDAGANDTSNNETNNGNDDNETENPNFWRQFGNRMQISPDQMRNLQMGLRNQNMVRNNNNNTIDESMPDTAIDSQMDEASGTPDEDML

(19) United States
(12) Patent Application Publication
(10) Pub. No.: US 2015/0299690 A1
(43) Pub. Date: Oct. 22, 2015

(54) ENGINEERED LOWER EUKARYOTIC HOST STRAINS DEFICIENT IN GRR1 ACTIVITY FOR RECOMBINANT PROTEIN

(71) Applicants: Bo JIANG, (US); Jun ZHUANG, (US); MERCK SHARP & DOHME CORP., Rahway, NJ (US)

(72) Inventors: Bo Jiang, Westfield, NJ (US); Jun Zhuang, Wellesley, MA (US)

(21) Appl. No.: 14/439,746

(22) PCT Filed: Dec. 5, 2013
(86) PCT No.: PCT/US13/73213

§ 371 Date: Apr. 30, 2015

Related U.S. Application Data
(60) Provisional application No. 61/735,325, filed on Dec. 10, 2012.

(51) Int. Cl. C12N 9/00; C12P 21/00

(52) U.S. Cl. CPC C12N 9/93; C12P 21/00; C12Y 603/02019

(57) ABSTRACT

The present invention relates to novel engineered lower eukaryotic host cells for expressing heterologous proteins and to methods of generating such strains. Lower eukaryotic host cells can be engineered to produce heterologous proteins. Further, lower eukaryotic host cells can be glyco-engineered to produce glycoproteins where the N- or O-linked glycosylation are modified from their native forms.

### FIELD OF THE INVENTION

The present invention relates to novel engineered lower eukaryotic host cells for expressing heterologous proteins and to methods of generating such strains.

### BACKGROUND OF THE INVENTION

Lower eukaryotic host cells can be engineered to produce heterologous proteins. Further, lower eukaryotic host cells can be glyco-engineered to produce glycoproteins where the N- or O-linked glycosylation are modified from their native forms.

Engineered Pichia strains have been utilized as an alternative host system for producing recombinant glycoproteins with human-like glycosylation. However, the extensive genetic modifications necessary to produce human-linke glycosylation have also caused fundamental changes in cell wall structures in many glyco-engineered yeast strains, predisposing some of these strains to cell lysis and reduced cell robustness during fermentation. Certain glyco-engineered strains have substantial reductions in cell viability as well as a marked increase in intracellular protease leakage into the fermentation broth, resulting in a reduction in both recombinant product yield and quality.

Current strategies for identifying robust glyco-engineered production strains rely heavily on screening a large number of clones using various platforms such as 96-deep-well plates, 5 ml mini-scale fermenters and 1 L-scale bioreactors to empirically identify clones that are compatible for large-scale (40 L and above) fermentation processes (Barnard et al. 2010). Despite the fact that high-throughput screening has been successfully used to identify several Pichia hosts capable of producing recombinant monoclonal antibodies with yields in excess of 1 g/L (Potgieter et al. 2009; Zhang et al. 2011), these large-scale screening approach is very resource-intensive and time-consuming, and often only identify clones with incremental increases in cell-robustness.

Therefore, lower eukaryotic host strains that have improved robustness and the ability to produce high quality proteins with human-like glycans would be of value and interest to the field. Here, we present engineered Pichia host strains having a deletion, truncation or nonsense mutation in a novel gene GRR1 which under relevant bioprocess conditions exhibit improved viability, stability, and protein production. Surprisingly, engineered Pichia host strains over-expressing GRR1 or fragments thereof under relevant bioprocess conditions also exhibit improved viability, stability, and protein production. These strains are especially useful for heterologous gene expression.

### SUMMARY OF THE INVENTION

The invention relates to engineered lower eukaryotic host cells that have a modified GRR1 gene. In one embodiment, the GRR1 gene has been modified by: (i) reducing or eliminating the expression of a GRR1 gene or polypeptide, or (ii) introducing a mutation in a GRR1 gene. In one embodiment, the GRR1 gene is modified by the introduction of a point mutation in the GRR1 gene. In one embodiment, the point mutation is at position 410, 451, 452 or 617 of SEQ ID NO:6. In one embodiment, the lower eukaryotic cell is a glyco-engineered lower eukaryotic host cells. In one embodiment, the lower eukaryotic cell is a lower eukaryotic host cell that lacks OCH1 activity. In one embodiment, the lower eukaryotic host cell is a fungal host cell. In one embodiment, the lower eukaryotic cell is a fungal host cell that lacks OCH1 activity. In one embodiment, the lower eukaryotic host cell is a yeast host cell. In one embodiment, the lower eukaryotic cell is a yeast host cell that lacks OCH1 activity. In one embodiment, the lower eukaryotic host cell is a Pichia sp. In one embodiment, the lower eukaryotic cell is a Pichia sp. host cell that lacks OCH1 activity. In one embodiment, the host cell is Pichia pastoris and the GRR1 gene encodes a polypeptide comprising the amino acid of SEQ ID NO:6 or a polymorph thereof. In another embodiment, the host cell is Pichia pastoris and the GRR1 gene encodes a polypeptide comprising the amino acid of SEQ ID NO:7. In another embodiment, the host cell is Pichia pastoris and the GRR1 gene encodes a polypeptide comprising the amino acid of SEQ ID NO:8. In another embodiment, the host cell is Pichia pastoris and the GRR1 gene encodes a polypeptide comprising the amino acid of SEQ ID NO:9. In another embodiment, the host cell is Pichia pastoris and the GRR1 gene encodes a polypeptide comprising the amino acid of SEQ ID NO:10. In one embodiment, the host cell is S. cerevisiae and the GRR1 gene encodes a polypeptide comprising the amino acid of SEQ ID NO:11.

In one embodiment, the GRR1 gene is modified to reduce or eliminate the activity of the GRR1 gene. The activity of the GRR1 gene can be reduced by any means. In one embodiment, the activity of the GRR1 gene is reduced or eliminated by reducing or eliminating the expression of the GRR1 gene (for example by using interfering RNA or antisense RNA). In another embodiment, the activity of the GRR1 gene is reduced or eliminated by mutating the GRR1 gene or its product. In another embodiment, the activity of the GRR1 gene is reduced or eliminated by degrading the GRR1 polypeptide. In another embodiment, the activity of the GRR1 gene is reduced or eliminated by using an inhibitor of GRR1, for example a small molecule inhibitor or an antibody inhibitor. The invention encompasses any means of inactivating the GRR1 gene or its protein including transcriptionally, translationally, or post-translationally means (for example, using repressible promoter, interfering RNA, antisense RNA, inducible protein degradation, and the like). In one embodiment, the lower eukaryotic cell is a glyco-engineered lower eukaryotic host cells. In one embodiment, the lower eukaryotic cell is a lower eukaryotic host cell that lacks OCH1 activity. In one embodiment, the lower eukaryotic host cell is a fungal host cell. In one embodiment, the lower eukaryotic cell is a fungal host cell that lacks OCH1 activity. In one embodiment, the lower eukaryotic host cell is a yeast host cell. In one embodiment, the lower eukaryotic cell is a yeast host cell that lacks OCH1 activity. In one embodiment, the lower eukaryotic host cell is a Pichia sp. In one embodiment, the lower eukaryotic cell is a Pichia sp. host cell that lacks OCH1 activity. In one embodiment, the host cell is Pichia pastoris and the GRR1 gene encodes a polypeptide comprising the amino acid of SEQ ID NO:6 or a polymorph thereof. In another embodiment, the host cell is Pichia pastoris and the GRR1 gene encodes a polypeptide comprising the amino acid of SEQ ID NO:7. In another embodiment, the host cell is Pichia pastoris and the GRR1 gene encodes a polypeptide comprising the amino acid of SEQ ID NO:8. In another embodiment, the host cell is Pichia pastoris and the GRR1 gene encodes a polypeptide comprising the amino acid of SEQ ID NO:9. In another embodiment, the host cell is Pichia pastoris and the GRR1 gene encodes a polypeptide comprising the amino acid of SEQ ID NO:10. In one embodiment, the host cell is S. cerevisiae and the GRR1 gene encodes a polypeptide comprising the amino acid of SEQ ID NO:11.

In other embodiments, the present invention relates to an engineered lower eukaryotic host cell that has been modified to express a mutated form of the GRR1 gene. The mutation could be a single nucleotide mutation, a frame-shift mutation, an insertion, a truncation or a deletion of one or more nucleotides. In one embodiment, said mutation is a deletion of the entire GRR1 gene. In another embodiment, said mutation is a deletion of a fragment of the GRR1 gene. In one embodiment, the lower eukaryotic cell is a glyco-engineered lower eukaryotic host cell. In one embodiment, the lower eukaryotic cell is a lower eukaryotic host cell that lacks OCH1 activity. In one embodiment, the lower eukaryotic host cell is a fungal host cell. In one embodiment, the lower eukaryotic cell is a fungal host cell that lacks OCH1 activity. In one embodiment, the lower eukaryotic host cell is a yeast host cell. In one embodiment, the lower eukaryotic cell is a yeast host cell that lacks OCH1 activity. In one embodiment, the lower eukaryotic host cell is a Pichia sp. In one embodiment, the lower eukaryotic cell is a Pichia sp. host cell that lacks OCH1 activity. In another embodiment, the host cell is Pichia pastoris and the GRR1 gene encodes a polypeptide comprising the amino acid of SEQ ID NO:6 or a polymorph thereof. In another embodiment, the host cell is Pichia pastoris and the GRR1 gene encodes a polypeptide comprising the amino acid of SEQ ID NO:7. In another embodiment, the host cell is Pichia pastoris and the GRR1 gene encodes a polypeptide comprising the amino acid of SEQ ID NO:8. In another embodiment, the host cell is Pichia pastoris and the GRR1 gene encodes a polypeptide comprising the amino acid of SEQ ID NO:9. In another embodiment, the host cell is Pichia pastoris and the GRR1 gene encodes a polypeptide comprising the amino acid of SEQ ID NO:10. In another embodiment, the host cell is Pichia pastoris and the mutated form of the GRR1 gene is an deletion, insertion or a frameshift mutation in the nucleic acid encoding SEQ ID NO:6. In another embodiment, the host cell is Pichia pastoris and the mutated form of the GRR1 gene is a single nucleotide mutation in the nucleic acid sequence encoding SEQ ID NO:6. In another embodiment, the host cell is Pichia pastoris and the mutated form of the GRR1 gene results in a single amino acid change in SEQ ID NO:6. In another embodiment, the host cell is Pichia pastoris and GRR1 gene comprises a mutation in the leucine rich repeat (amino acids 155-471 of SEQ ID NO:6). In one embodiment, the host cell is S. cerevisiae and the GRR1 gene encodes a polypeptide comprising the amino acid of SEQ ID NO:11. In another embodiment, the host cell is S. cerevisiae and the mutated form of the GRR1 gene is an deletion, insertion or a frameshift mutation in the nucleic acid encoding SEQ ID NO:11. In another embodiment, the host cell is S. cerevisiae and the mutated form of the GRR1 gene is a single nucleotide mutation in the nucleic acid sequence encoding SEQ ID NO:11. In another embodiment, the host cell is S. cerevisiae and mutated form of the GRR1 gene results in a single amino acid change in SEQ ID NO:11.

In some embodiments, the engineered lower eukaryotic host cell of the invention exhibits an increase in culture stability, thermal tolerance and/or improved fermentation robustness compared with a GRR1 naïve parental host cell under similar culture conditions. In one embodiment, said engineered host cell is capable of surviving in culture at 32° C. for at least 80 hours of fermentation with minimal cell lysis. In one embodiment, said engineered host cell is capable of surviving in culture at 32° C. for at least 80 hours of fermentation after induction (for example, methanol induction) with minimal cell lysis. In one embodiment, said engineered host cell is capable of surviving in culture at 32° C. for at least 100 hours of fermentation with minimal cell lysis. In one embodiment, said engineered host cell is capable of surviving in culture at 32° C. for at least 100 hours of fermentation after induction with minimal cell lysis.

In some embodiments, the engineered lower eukaryotic host cell of the invention further comprises a mutation, disruption or deletion of one or more of functional gene products. In one embodiment, the host cell comprises a mutation, disruption or deletion of one or more genes encoding: protease activities, alpha-1,6-mannosyltransferase activities, alpha-1,2-mannosyltransferase activities, mannosylphosphate transferase activities, (3-mannosyltransferase activities, 0-mannosyltransferase (PMT) activities, and/or dolichol-β-Man dependent alpha(1-3) mannosyltransferase activities. In one embodiment, the host cell comprises a mutation, disruption or deletion in the OCH1 gene. In one embodiment, the host cell comprises a mutation, disruption or deletion in the BMT1, BMT2, BMT3, and BMT4 genes. In one embodiment, the host cell comprises a mutation, disruption or deletion in the PNO1, MNN4, and MNN4L1 genes. In one embodiment, the host cell comprises a mutation, disruption or deletion in the PEP4 and PRB1 genes. In another embodiment, the host cell comprises a mutation, disruption or deletion of the ALG3 gene (as described in US Patent Publication No. US2005/0170452). In one embodiment, the host cell further comprises a mutation, disruption or deletion of all of the following genes: OCH1, BMT1, BMT2, BMT3, BMT4, PNO1, MNN4, and MNN4L1. In one embodiment, the host cell further comprises a mutation, disruption or deletion of all of the following genes: OCH1, BMT1, BMT2, BMT3, BMT4, PNO1, MNN4, MNN4L1, PEP4 and PRB1. In one embodiment, the host cell further comprises a mutation, disruption or deletion of all of the following genes: OCH1, BMT1, BMT2, BMT3, BMT4, PNO1, MNN4, MNN4L1, ALG3, PEP4 and PRB1. In one embodiment, the engineered lower eukaryotic host cell of the invention further comprises a mutation, disruption or deletion of a gene selected from the group consisting of: CRZ1 and ATT1.

In yet additional embodiments, the engineered lower eukaryotic host cell of the invention further comprises one or more nucleic acid sequences of interest. In certain embodiments, the nucleic acid sequences of interest encode one or more glycosylation enzymes. In certain embodiments, the glycosylation enzymes are selected from the group consisting of glycosidases, mannosidases, phosphomannosidases, phosphatases, nucleotide sugar transporters, nucleotide sugar epimerases, mannosyltransferases, N-acetylglucosaminyltransferases, CMP-sialic acid synthases, N-acetylneuraminate-9-phosphate synthases, galactosyltransferases, sialyltransferases, and oligosaccharyltransferases. In yet additional embodiments, the engineered lower eukaryotic host cell of the invention further comprises a nucleic acid sequences encoding one or more recombinant proteins. In one embodiment, the recombinant protein is a therapeutic protein. The therapeutic protein can contain or lack oligosaccharides. In certain embodiments, the therapeutic proteins are selected from the group consisting of antibodies (IgA, IgG, IgM or IgE), antibody fragments, kringle domains of the human plasminogen, erythropoietin, cytokines, coagulation factors, soluble IgE receptor α-chain, urokinase, chymase, urea trypsin inhibitor, IGF-binding protein, epidermal growth factor, growth hormone-releasing factor, annexin V fusion protein, angiostatin, vascular endothelial growth factor-2, myeloid progenitor inhibitory factor-1, osteoprotegerin, α-1 antitrypsin, DNase II, α-feto proteins, insulin, Fc-fusions, HSA-fusions, viral antigens and bacterial antigens. In one embodiment, the therapeutic protein is an antibody or a fragment thereof. In one embodiment, the therapeutic protein is an antibody or antibody fragment (Fc-containing polypeptide) comprising N-glycans. In one embodiment, the N-glycans comprise predominantly NANA₍₁₋₄₎Gal₍₁₋₄₎Man₃GlcNAc₂. In one embodiment, the N-glycans comprise predominantly NANA₂Gal₂Man₃GlcNAc₂.

In certain embodiments, the invention also provides engineered lower eukaryotic host cells comprising a disruption, deletion or mutation (e.g., a single nucleotide mutation, insertion mutation, or deletion mutation) of a nucleic acid sequence selected from the group consisting of: the coding sequence of the GRR1 gene, the promoter region of the GRR1 gene, the 3′ un-translated region (UTR) of GRR1, a nucleic acid sequence that is a degenerate variant of the coding sequence of the P. pastoris GRR1 gene and related nucleic acid sequences and fragments, in which the host cells have an increase in culture stability, thermal tolerance or improved fermentation robustness compared to a host cell without the disruption, deletion or mutation.

The invention also relates to methods of using the engineered lower eukaryotic host cells of the invention for producing heterologous polypeptides and other metabolites. In one embodiment, the invention provides for methods for producing a heterologous polypeptide in any of the Pichia sp. host cells described above comprising culturing said host cell under conditions favorable to the expression of the heterologous polypeptide; and, optionally, isolating the heterologous polypeptide from the host cell.

The invention also comprises a method for producing a heterologous polypeptide in an engineered lower eukaryotic host cell, said method comprising: (a) introducing a polynucleotide encoding a heterologous polypeptide into an engineered host cell which has been modified to reduce or eliminate the activity of a GRR1 gene which is an ortholog to the Pichia pastoris GRR1 gene; (b) culturing said host cell under conditions favorable to the expression of the heterologous polypeptide; and, optionally, (c) isolating the heterologous polypeptide from the host cell. In one embodiment, the lower eukaryotic host cell is glyco-engineered. In one embodiment, the lower eukaryotic cell lacks OCH1 activity. In one embodiment, the lower eukaryotic host cell is a fungal host cell. In one embodiment, the lower eukaryotic cell is a fungal host cell that lacks OCH1 activity. In one embodiment, the lower eukaryotic host cell is a yeast host cell. In one embodiment, the lower eukaryotic cell is a yeast host cell that lacks OCH1 activity. In one embodiment, the lower eukaryotic host cell is a Pichia sp. In one embodiment, the lower eukaryotic cell is a Pichia sp. host cell that lacks OCH1 activity. In another embodiment, the host cell is Pichia pastoris and the GRR1 gene encodes a polypeptide comprising the amino acid of SEQ ID NO:6 or a polymorph thereof.

The invention also provides a method for making any of the host cells of the invention, comprising introducing a heterologous polynucleotide into the cell which homologously recombines with the endogenous GRR1 gene and partially or fully deletes the endogenous GRR1 gene or disrupts the endogenous GRR1 gene.

In addition, the invention provides methods for the genetic integration of a heterologous nucleic acid sequence into a host cell comprising a disruption, deletion or mutation of the GRR1 gene in the genomic DNA of the host cell. These methods comprise the step of introducing a sequence of interest into the host cell comprising a disrupted, deleted or mutated nucleic acid sequence derived from a sequence selected from the group consisting of the coding sequence of the P. pastoris GRR1 gene, a nucleic acid sequence that is a degenerate variant of the coding sequence of the P. pastoris GRR1 gene and related nucleic acid sequences and fragments.

The invention also provides isolated polynucleotides encoding the P. pastoris GRR1 gene, or a fragment of the P. pastoris GRR1 gene, or an ortholog or polymorph (natural variant) of the P. pastoris GRR1 gene. The invention also provides isolated polynucleotides encoding mutants of the GRR1 gene (single nucleotide mutations, frame-shift mutations, insertions, truncations or deletions). The invention also provides vectors and host cells comprising these isolated polynucleotides or fragments of these polynucleotides. The invention further provides isolated polypeptides comprising or consisting of the polypeptide sequence encoded by the P. pastoris GRR1 gene, by a fragment of the P. pastoris GRR1 gene, or an ortholog or polymorph of the P. pastoris GRR1 gene. Antibodies that specifically bind to the isolated polypeptides of the invention are also encompassed herein.

In one embodiment, the invention comprises an expression vector comprising a nucleic acid encoding a wild-type or mutated GRR1 gene selected from the group consisting of: a nucleotide sequence encoding SEQ ID NO:6 or a fragment thereof; a nucleotide sequence encoding SEQ ID NO:7 or a fragment thereof; a nucleotide sequence encoding SEQ ID NO:8 or a fragment thereof; a nucleotide sequence encoding SEQ ID NO:9 or a fragment thereof; and a nucleotide sequence encoding SEQ ID NO:10 or a fragment thereof. In one embodiment, an isolated host cell expressing said nucleic acid exhibits an increase in culture stability, thermal tolerance and/or improved fermentation robustness compared to a GRR1 naive parental host cell under similar conditions. The invention also comprises vectors and host cells comprising the nucleic acids of the invention, and the polypeptides encoded by these nucleic acids.

### BRIEF DESCRIPTION OF THE DRAWINGS

FIG. 1 shows a strain lineage for four Pichia pastoris GRR1 mutant stains.

FIG. 2 shows the improved fermentation robustness of GRR1 mutant strains.

FIG. 3 depicts a diagram of the GRR1 gene mutations of the identified GRR1 mutants.

FIG. 4 shows that GRR1 mutant strains display similar product titers that wild type strains.

FIG. 5 shows that GRR1 mutant strains product glycoproteins having similar N-glycan compositions as the glycoproteins produced in wild type strains.

### DETAILED DESCRIPTION OF THE INVENTION

### Molecular Biology

In accordance with the present invention there may be employed conventional molecular biology, microbiology, and recombinant DNA techniques within the skill of the art. Unless otherwise defined herein, scientific and technical terms used in connection with the present invention shall have the meanings that are commonly understood by those of ordinary skill in the art. Further, unless otherwise required by context, singular terms shall include the plural and plural terms shall include the singular. Generally, nomenclatures used in connection with, and techniques of biochemistry, enzymology, molecular and cellular biology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. See, e.g., James M. Cregg (Editor), Pichia Protocols (Methods in Molecular Biology), Humana Press (2010), Sambrook et al. Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lane, Antibodies: A Laboratory Manual, Cold Spring Harbor Laboratory Press, Cold Spring Harbor, N.Y. (1990); Taylor and Drickamer, Introduction to Glycobiology, Oxford Univ. Press (2003); Worthington Enzyme Manual, Worthington Biochemical Corp., Freehold, N.J.; Handbook of Biochemistry: Section A Proteins, Vol I, CRC Press (1976); Handbook of Biochemistry: Section A Proteins, Vol II, CRC Press (1976); Essentials of Glycobiology, Cold Spring Harbor Laboratory Press (1999), Animal Cell Culture (R. I. Freshney, ed. (1986)); Immobilized Cells And Enzymes (IRL Press, (1986)); B. Perbal, A Practical Guide To Molecular Cloning (1984).

A “polynucleotide” and “nucleic acid” includes DNA and RNA in single stranded form, double-stranded form or otherwise.

A “polynucleotide sequence” or “nucleotide sequence” is a series of nucleotide bases (also called “nucleotides”) in a nucleic acid, such as DNA or RNA, and means a series of two or more nucleotides. Any polynucleotide comprising a nucleotide sequence set forth herein (e.g., promoters of the present invention) forms part of the present invention.

A “coding sequence” or a sequence “encoding” an expression product, such as an RNA or polypeptide is a nucleotide sequence (e.g., heterologous polynucleotide) that, when expressed, results in production of the product (e.g., a polypeptide comprising SEQ ID NO:6 or a fragment of SEQ ID NO:6).

A “protein”, “peptide” or “polypeptide” (e.g., a heterologous polypeptide such SEQ ID NO:6 or as an immunoglobulin heavy chain and/or light chain) includes a contiguous string of two or more amino acids.

A “protein sequence”, “peptide sequence” or “polypeptide sequence” or “amino acid sequence” refers to a series of two or more amino acids in a protein, peptide or polypeptide.

The term “isolated polynucleotide” or “isolated polypeptide” includes a polynucleotide or polypeptide, respectively, which is partially or fully separated from other components that are normally found in cells or in recombinant DNA expression systems or any other contaminant. These components include, but are not limited to, cell membranes, cell walls, ribosomes, polymerases, serum components and extraneous genomic sequences. The scope of the present invention includes the isolated polynucleotides set forth herein, e.g., the promoters set forth herein; and methods related thereto, e.g., as discussed herein.

An isolated polynucleotide or polypeptide will, preferably, be an essentially homogeneous composition of molecules but may contain some heterogeneity.

In general, a “promoter” or “promoter sequence” is a DNA regulatory region capable of binding an RNA polymerase in a cell (e.g., directly or through other promoter-bound proteins or substances) and initiating transcription of a coding sequence to which it operably links.

A coding sequence (e.g., of a heterologous polynucleotide, e.g., reporter gene or immunoglobulin heavy and/or light chain) is “operably linked to”, “under the control of”, “functionally associated with” or “operably associated with” a transcriptional and translational control sequence (e.g., a promoter of the present invention) when the sequence directs RNA polymerase mediated transcription of the coding sequence into RNA, preferably mRNA, which then may be RNA spliced (if it contains introns) and, optionally, translated into a protein encoded by the coding sequence.

The present invention includes vectors or cassettes which comprise a nucleic acid encoding a wildtype GRR1 or a mutated GRR1 coding region (including single nucleotide mutations, frameshift mutations, insertions, truncations and deletions in the GRR1 gene). The present invention also includes vectors that lead to over-expression of GRR1 or a fragment of GRR1 which is able to increase culture stability, thermal tolerance, and/or improved fermentation robustness when overexpressed. The term “vector” includes a vehicle (e.g., a plasmid) by which a DNA or RNA sequence can be introduced into a host cell, so as to transform the host and, optionally, promote expression and/or replication of the introduced sequence. Suitable vectors for use herein include plasmids, integratable DNA fragments, and other vehicles that may facilitate introduction of the nucleic acids into the genome of a host cell (e.g., Pichia pastoris). Plasmids are the most commonly used form of vector but all other forms of vectors which serve a similar function and which are, or become, known in the art are suitable for use herein. See, e.g., Pouwels, et al., Cloning Vectors: A Laboratory Manual, 1985 and Supplements, Elsevier, N.Y., and Rodriguez et al. (eds.), Vectors: A Survey of Molecular Cloning Vectors and Their Uses, 1988, Buttersworth, Boston, Mass.

A polynucleotide (e.g., a heterologous polynucleotide, e.g., encoding an immunoglobulin heavy chain and/or light chain), operably linked to a promoter, may be expressed in an expression system. The term “expression system” means a host cell and compatible vector which, under suitable conditions, can express a protein or nucleic acid which is carried by the vector and introduced to the host cell. Common expression systems include fungal host cells (e.g., Pichia pastoris) and plasmid vectors, insect host cells and Baculovirus vectors, and mammalian host cells and vectors.

In general, “inducing conditions” refer to growth conditions which result in an enhanced expression of a polynucleotide (e.g. a heterologous polynucleotide) in a host cell. The term methanol-induction refers to increasing expression of a polynucleotide (e.g., a heterologous polynucleotide) operably linked to a methanol-inducible promoter in a host cell of the present invention by exposing the host cells to methanol.

The following references regarding the BLAST algorithm are herein incorporated by reference: BLAST ALGORITHMS: Altschul, S. F., et al., J. Mol. Biol. (1990) 215:403-410; Gish, W., et al., Nature Genet. (1993) 3:266-272; Madden, T. L., et al., Meth. Enzymol. (1996) 266:131-141; Altschul, S. F., et al., Nucleic Acids Res. (1997) 25:3389-3402; Zhang, J., et al., Genome Res. (1997) 7:649-656; Wootton, J. C., et al., Comput. Chem. (1993) 17:149-163; Hancock, J. M., et al., Comput. Appl. Biosci. (1994) 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., J. Mol. Biol. (1991) 219:555-565; States, D. J., et al., Methods (1991) 3:66-70; Henikoff, S., et al., Proc. Natl. Acad. Sci. USA (1992)89:10915-10919; Altschul, S. F., et al., J. Mol. Evol. (1993) 36:290-300; ALIGNMENT STATISTICS: Karlin, S., et al., Proc. Natl. Acad. Sci. USA (1990) 87:2264-2268; Karlin, S., et al., Proc. Natl. Acad. Sci. USA (1993) 90:5873-5877; Dembo, A., et al., Ann. Prob. (1994) 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

### Host Cells

The invention relates to engineered lower eukaryotic host cells that have been modified to reduce or eliminate the activity of the GRR1 gene. In one embodiment, the lower eukaryotic host cell is glyco-engineered. In one embodiment, the lower eukaryotic host cell lacks OCH1 activity. In one embodiment, the lower eukaryotic host cell is a fungal host cell. In one embodiment, the lower eukaryotic host cell is a fungal host cell that lacks OCH1 activity. In another embodiment, the lower eukaryotic host cell host cell is a yeast host cell. In another embodiment, the lower eukaryotic host cell host cell is a yeast host cell that clacks OCH1 activity. In one embodiment, the lower eukaryotic host cell is a Pichia sp. In one embodiment, lower eukaryotic host cell is a Pichia sp. that lacks OCH1 activity. In one embodiment, the fungal host cell is selected from the group consisting of: Pichia pastoris, Pichia angusta (Hansenula polymorpha), Pichia finlandica, Pichia trehalophda, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Yarrowia Lipolytica, Kluyveromyces lactis, Zygosaccharomyces rouxii, Zygosaccharomyces bailii, Schwanniomyces occidentalis, Kluyveromyces marxianus, Aspergillus niger, Arxula adeninivorans, Aspergillus nidulans, Aspergillus wentii, Aspergillus aureus, Aspergillus flavus, Ashbya gossypii, Methylophdus methylotrophus, Schizosaccharomyces pombe, Candida boidinii, Candida utilis, Rhizopus oryzae, Debaromyces hansenii and Saccharyomyces cerevisiae. In another embodiment, the fungal host cell is Pichia pastoris.

As used herein, a host cell which has reduced GRR1 gene activity or lacks GRR1 gene activity refers to a cell that has an increase in culture stability, thermal tolerance and/or improved fermentation robustness compared with a GRR1 naïve parental host cell under similar culture conditions. In order to determine if a gene has GRR1 activity, the gene can be deleted in a glyco-engineered host cell (for example, an OCH1 minus lower eukaryotic host cell) and the ability of the cell (with the GRR1 gene deletion) to survive in culture at 32° C. within a bioreactor is determined, if the cell has increased culture stability, thermal tolerance and/or improved robustness compared to a GRR1 naïve cell then the gene has GRR1 activity.

As used herein, a “GRR1 naïve host cell” refers to a host cell that comprises a wild-type GRR1 gene in its native genomic state. For example, in one embodiment, a GRR1 naïve host cell refers to a Pichia pastoris strain comprising in its native genomic state a GRR1 gene encoding the polypeptide of SEQ ID N0:6 or a natural variant (polymorph) thereof.

As used herein, an “engineered cell” refers to cell that has been altered using genetic engineering techniques. As used herein, a “glyco-engineered” cell refers to cell that has been genetically engineered to produce glycoproteins where the N- or O-linked glycosylation are modified from their native form, either through inactivation or deletion of genes or through the heterologous expression of glycosyltransferases or glycosidases.

As used herein “thermal tolerance” refers to increase in temperature resistance (i.e. ability to grow in culture to temperatures of at least about 32° C.).

As used herein, “improved fermentation robustness” refers to an increase in cell viability or decrease in cell lysis during fermentation.

The invention encompasses any engineered lower eukaryotic host cell which has been modified to: reduce or eliminate the activity of an GRR1 gene which is an ortholog of the Pichia pastoris GRR1 gene; wherein the cell exhibits an increase in culture stability, thermal tolerance, and/or improved fermentation robustness when compared to an GRR1 naïve parental host cell.

The invention also relates to an engineered lower eukaryotic host cell which has been modified to (i) reduce or eliminate expression of an GRR1 gene or polypeptide which is an ortholog of the Pichia pastoris GRR1 gene, or (ii) express a mutated form of an GRR1 gene which is an ortholog of the Pichia pastoris GRR1 gene; wherein said cell exhibits an increase in culture stability, thermal tolerance, and/or improved fermentation robustness when compared to an GRR1 naïve parental host cell. In one embodiment, the invention relates to an engineered lower eukaryotic host cell which has been modified to reduce or eliminate expression of an GRR1 gene or polypeptide which is an ortholog of the Pichia pastoris GRR1 gene or to express a mutated form of an GRR1 gene which is an ortholog of the Pichia pastoris GRR1 gene; wherein said cell exhibits an increase in culture stability, thermal tolerance, and/or improved fermentation robustness when compared to an GRR1 naïve parental host cell.

As used herein, an ortholog to the Pichia pastoris GRR1 gene, is a gene that has sequence similarity to the Pichia pastoris GRR1 gene and has GRR1 activity. In one embodiment, the sequence similarity will be at least 25%. A person of skill in the art would be able to identify such orthologs using only routine experimentation. Other fungal/yeast orthologs could be similarly identified, for example by the use of reciprocal BLAST analysis.

The host cells of the invention could be in haploid, diploid, or polyploid state. Further, the invention encompasses a diploid cell wherein only one endogenous chromosomal GRR1 gene has been mutated, disrupted, truncated or deleted.

In one embodiment, the engineered lower eukaryotic host cell of the invention is selected from the group consisting of: Pichia pastoris, Pichia angusta (Hansenula polymorpha), Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Yarrowia Lipolytica, Kluyveromyces lactis, Zygosaccharomyces rouxii, Zygosaccharomyces bailii, Schwanniomyces occidentalis, Kluyveromyces marxianus, Aspergillus niger, Arxula adeninivorans, Aspergillus nidulans, Aspergillus wentii, Aspergillus aureus, Aspergillus flavus, Ashbya gossypii, Methylophilus methylotrophus, Schizosaccharomyces pombe, Candida boidinii, Candida utilis, Rhizopus oryzae and Debaromyces hansenii. In an embodiment of the invention, the host cell is selected from the group consisting of any Pichia cell, such as Pichia pastoris, Pichia angusta (Hansenula polymorpha), Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, and Pichia methanolica. In one embodiment, the host cell is an engineered Pichia pastoris host cell and the GRR1 gene encodes a polypeptide comprising the amino acid sequence of SEQ ID NO:6 or a natural variant of said polypeptide.

In one embodiment, the engineered lower eukaryotic host cells of the invention further comprise a mutation, disruption or deletion of one or more of genes. In one embodiment, the engineered lower eukaryotic host cell of the invention comprises a mutation, disruption or deletion of one or more genes encoding protease activities, alpha-1,6-mannosyltransferase activities, alpha-1,2-mannosyltransferase activities mannosylphosphate transferase activities, β-mannosyltransferase activities, O-mannosyltransferase (PMT) activities, and/or dolichol-P-Man dependent alpha(1-3) mannosyltransferase activities. In one embodiment, an engineered lower eukaryotic host cell of the invention comprises a mutation, disruption or deletion in the OCH1 gene. In one embodiment, an engineered lower eukaryotic host cell of the invention comprises a mutation, disruption or deletion in the BMT1, BMT2, BMT3, and BMT4 genes. In one embodiment, an engineered lower eukaryotic host cell of the invention comprises a mutation, disruption or deletion in the PNO1, MNN4, and MNN4L1 genes. In one embodiment, an engineered lower eukaryotic host cell of the invention comprises a mutation, disruption or deletion in the PEP4 and PRB1 genes. In another embodiment, an engineered lower eukaryotic host cell of the invention comprises a mutation, disruption or deletion of the ALG3 gene (as described in US Patent Publication No. US2005/0170452). In one embodiment, an engineered lower eukaryotic host cell of the invention comprises a mutation, disruption or deletion of all of the following genes: OCH1, BMT1, BMT2, BMT3, BMT4, PNO1, MNN4, and MNN4L1. In one embodiment, an engineered lower eukaryotic host cell of the invention comprises a mutation, disruption or deletion of all of the following genes: OCH1, BMT1, BMT2, BMT3, BMT4, PNO1, MNN4, MNN4L1, PEP4 and PRB1. In one embodiment, an engineered lower eukaryotic host cell of the invention comprises a mutation, disruption or deletion of all of the following genes: OCH1, BMT1, BMT2, BMT3, BMT4, PNO1, MNN4, MNN4L1, ALG3, PEP4 and PRB1.

In some embodiments, the host cell of the invention can be cultivated in a medium that includes one or more Pmtp inhibitors. Pmtp inhibitors include but are not limited to a benzylidene thiazolidinedione. Examples of benzylidene thiazolidinediones are 5-[[3,4bis(phenylmethoxy)phenyl]methylene]-4-oxo-2-thioxo-3-thiazolidineacetic Acid; 5-[[3-(1-25 Phenylethoxy)-4-(2-phenylethoxy)]phenyl]methylene]-4-oxo-2-thioxo-3-thiazolidineacetic Acid; and 5-[[3-(1-Phenyl-2-hydroxy)ethoxy)-4-(2-phenylethoxy)]phenyl]methylene]-4-oxo-2-thioxo3-thiazolidineacetic acid.

In one embodiment, an engineered lower eukaryotic host cell of the invention lacks OCH1 activity. In one embodiment, the invention comprises a lower eukaryotic host cell (e.g., Pichia sp.) that has been modified to: (i) reduce or eliminate expression of a GRR1 gene or polypeptide, or (ii) express a mutated form of a GRR1 gene, wherein the cell lacks OCH1 activity. Lower eukaryotic cells lacking OCH1 activity have been described in the art and have been shown to be temperature sensitive. See, e.g., Choi et al., 2003; Bates et al., J. Biol. Chem. 281(1):90-98 (2006); Woog Kim et al., J. Biol. Chem. 281(10):6261-6272 (2006); Yoko-o et al., FEBS Letters 489(1):75-80 (2001); and Nakayama et al., EMBO J 11(7):2511-2519 (1992). Accordingly, it is desirable to modify cells that lack OCH1 activity to render them thermotolerant.

In an embodiment of the invention, an engineered lower eukaryotic host cell of the invention is further genetically engineered to include a nucleic acid that encodes an alpha-1,2-mannosidase that has a signal peptide that directs it for secretion. For example, in an embodiment of the invention, the host cell of the invention is engineered to express an exogenous alpha-1,2-mannosidase enzyme having an optimal pH between 5.1 and 8.0, preferably between 5.9 and 7.5. In an embodiment of the invention, the exogenous enzyme is targeted to the endoplasmic reticulum or Golgi apparatus of the host cell, where it trims N-glycans such as Man₈GlcNAc₂ to yield Man₅GlcNAc₂. See U.S. Pat. No. 7,029,872. Lower eukaryotic host cells expressing such alpha-1,2-mannosidase activity have been described in the art, see, e.g., Choi et al., 2003. In one embodiment, the glyco-engineered lower eukaryotic host cell of the invention lacks OCH1 activity and comprises an alpha1,2 mannosidase.

In another embodiment, engineered lower eukaryotic host cells (e.g., Pichia sp.) of the invention that have been modified to: (i) reduce or eliminate expression of an GRR1 gene or polypeptide, or (ii) express a mutated form of an GRR1 gene, are further genetically engineered to eliminate glycoproteins having alpha-mannosidase-resistant N-glycans by deleting or disrupting one or more of the beta-mannosyltransferase genes (e.g., BMT1, BMT2, BMT3, and BMT4) (See, U.S. Pat. No. 7,465,577) or abrogating translation of RNAs encoding one or more of the beta-mannosyltransferases using interfering RNA, antisense RNA, or the like.

In some embodiments, engineered lower eukaryotic host cells (e.g., Pichia sp.) of the present invention that have been modified to: (i) reduce or eliminate expression of an GRR1 gene or polypeptide or (ii) express a mutated form of an GRR1 gene, are further genetically engineered to eliminate glycoproteins having phosphomannose residues, e.g., by deleting or disrupting one or more of the phosphomannosyl transferase genes (i.e., PNO1, MNN4 and MNN4L1 (see e.g., U.S. Pat. Nos. 7,198,921 and 7,259,007)), or by abrogating translation of RNAs encoding one or more of the phosphomannosyltransferases using interfering RNA, antisense RNA, or the like.

Additionally, engineered lower eukaryotic host cells (e.g., Pichia sp.) of the invention that have been modified to: (i) reduce or eliminate expression of an GRR1 gene or polypeptide or (ii) express a mutated form of an GRR1 gene, may be further genetically engineered to include a nucleic acid that encodes the Leishmania sp. single-subunit oligosaccharyltransferase STT3A protein, STT3B protein, STT3C protein, STT3D protein, or combinations thereof such as those described in WO2011/06389.

In some embodiments, the engineered lower eukaryotic host cell of the invention further comprises a promoter operably linked to a polynucleotide encoding a heterologous polypeptide (e.g., a reporter or immunoglobulin heavy and/or light chain). The invention further comprises methods of using the host cells of the invention, e.g., methods for expressing the heterologous polypeptide in the host cell. The engineered lower eukaryotic host cell of the invention may be also genetically engineered so as to express particular glycosylation patterns on polypeptides that are expressed in such cells. For example, host cells of the present invention may be modified to produce polypeptides comprising N-glycans. In one embodiment, the host cells of the invention may be engineered to produce high mannose, hybrid or complex-type N-glycans.

As used herein, the terms “N-glycan” and “glycoform” are used interchangeably and refer to an N-linked oligosaccharide, e.g., one that is attached by an asparagine-N-acetylglucosamine linkage to an asparagine residue of a polypeptide. N-linked glycoproteins contain an N-acetylglucosamine residue linked to the amide nitrogen of an asparagine residue in the protein. Predominant sugars found on glycoproteins are glucose, galactose, mannose, fucose, N-acetylgalactosamine (GalNAc), N-acetylglucosamine (GlcNAc) and sialic acid (e.g., N-acetyl-neuraminic acid (NANA)).

N-glycans have a common pentasaccharide core of Man₃GlcNAc₂ (“Man” refers to mannose; “Glc” refers to glucose; and “NAc” refers to N-acetyl; GlcNAc refers to N-acetylglucosamine)N-glycans differ with respect to the number of branches (antennae) comprising peripheral sugars (e.g., GlcNAc, galactose, fucose and sialic acid) that are added to the Man₃GlcNAc₂ (“Man₃”) core structure which is also referred to as the “trimannose core”, the “pentasaccharide core” or the “paucimannose core”. N-glycans are classified according to their branched constituents (e.g., high mannose, complex or hybrid). A “high mannose” type N-glycan has five or more mannose residues. A “complex” type N-glycan typically has at least one GlcNAc attached to the 1,3 mannose arm and at least one GlcNAc attached to the 1,6 mannose arm of a “trimannose” core. Complex N-glycans may also have galactose (“Gal”) or N— acetylgalactosamine (“GalNAc”) residues that are optionally modified with sialic acid or derivatives (e.g., “NANA” or “NeuAc”, where “Neu” refers to neuraminic acid and “Ac” refers to acetyl). Complex N-glycans may also have intrachain substitutions comprising “bisecting” GlcNAc and core fucose (“Fuc”). Complex N-glycans may also have multiple antennae on the “trimannose core,” often referred to as “multiple antennary glycans.” A “hybrid” N-glycan has at least one GlcNAc on the terminal of the 1,3 mannose arm of the trimannose core and zero or more mannoses on the 1,6 mannose arm of the trimannose core. The various N-glycans are also referred to as “glycoforms”. “PNGase” or “glycanase” refers to peptide N-glycosidase F (EC 3.2.2.18).

In an embodiment of the invention, engineered lower eukaryotic host cells (e.g., Pichia sp.) of the invention that have been modified to: (i) reduce or eliminate expression of an GRR1 gene or polypeptide or (ii) express a mutated form of an GRR1 gene, are further genetically engineered to produce glycoproteins that have predominantly an N-glycan selected from the group consisting of complex N-glycans, hybrid N-glycans, and high mannose N-glycans. In one embodiment, the high mannose N-glycans are selected from the group consisting of Man₆GlcNAc₂, Man₇GlcNAc₂, Man₈GlcNAc₂, and Man₉GlcNAc₂. In one embodiment, the host cell of the invention is engineered to produce glycoproteins that have predominantly Man₈₋₁₀GlcNAc₂ N-glycans. In one embodiment, the N-glycans are selected from the group consisting of Man₅GlcNAc₂, GlcNAcMan₅GlcNAc₂, GalGlcNAcMan₅GlcNAc₂, and NANAGalGlcNAcMan₅GlcNAc₂. In one embodiment, the N-glycans are selected from the group consisting of Man₃GlcNAc₂, GlcNAC₍₁₋₄₎Man₃GlcNAc₂, NANA₍₁₋₄₎GlcNAc₍₁₋₄₎Man₃GlcNAc₂, and NANA₍₁₋₄₎Gal₍₁₋₄₎Man₃GlcNAc_{]}. In one embodiment, the N-glycans comprise predominantly a Man₃GlcNAc₂ structure. In one embodiment, the N-glycans comprise predominantly NANA₍₁₋₄₎Gal₍₁₋₄₎Man₃GlcNAc₂. In one embodiment, the N-glycans comprise predominantly NANA₂Gal₂Man₃GlcNAc₂. In one embodiment, the host cell of the invention is engineered to produce glycoproteins that have galactosylated N-glycans. In one embodiment, the host cell of the invention is engineered to produce glycoproteins that have sialylated N-glycans (WO2011/149999).

Characterization of Pichia pastoris GRR1

This invention describes the identification of mutations within a Pichia pastoris gene GRR1, a homolog of S. cerevisiae's GRR1 which is a F-box protein component of the SCF ubiquitin-ligase complex. Mutations in the GRR1 protein led to a significant enhancement in thermal tolerance and fermentation robustness in Pichia pastoris strains. The GRR1 mutations described in this application could be engineered into any Pichia host strain for the purposes of increasing fermentation robustness, improving recombinant protein yield, and reducing product proteolytic degradation.

Further, GRR1 mutant Pichia strains exhibited decreased lysis, extended induction/production phase, and produced heterologous protein products with decreased proteolytic degradation as well as desired glycosylation patterns. While non-mutagenized glyco-engineered parental strains typically display a temperature-sensitive phenotype when grown on Petri dishes (Choi et al. 2003) and generally display a high level of cell lysis within 40 to 50 hours of MeOH induction at 32° C. when cultured within a bioreactor, the GRR1 mutant strains described herein are viable for more than 80 hours after induction at 32° C. when cultured within a bioreactor, without showing obvious signs of cell-lysis. This extended induction period allows for significantly increased yield and quality of multiple recombinant proteins, desirable traits for production of heterologous proteins such as antibody and non-antibody therapeutics.

### Experimental Methods

Fed-batch fermentations, IgG purifications, N-glycan characterizations, as well as all other analytical assays, were performed as previously described (Barnard et al. 2010; Jiang et al. 2011; Potgieter et al. 2009; Winston F 2008). UV mutagenesis was performed as described by Winston (Winston 2008). Briefly, Pichia strains were grown in 40 ml YSD liquid medium overnight at 24° C. Upon reaching an OD₆₀₀ of 5, an aliquot of 10⁶ to 10⁷ cells was transferred onto the surface of a 100 mm YSD agar Petri dish, and treated, with the lid off, with 5 mJ/cm² of UV irradiation. After the UV treatment, the Petri dish was immediately covered with aluminum foil (to prevent photo-induced DNA repair) and the mutagenized cells were allowed to recover at 24° C. for 18 hours in the dark. Then, these recovered cells were transferred to 35° C. incubator to select for temperature-resistant mutants. After 7-10 days incubation at 35° C., colonies were picked and re-streaked onto fresh YSD plates and incubated at 35° C., and only the clones displaying the temperature-resistant phenotype upon restreak were retained as temperature-resistant mutants.

### Example 1

### Temperature-Resistant Mutants Displayed Substantially Enhanced Fermentation Robustness and Productivity

To identify Pichia host strains with increased fermentation robustness, we UV-mutagenized two temperature-sensitive glyco-engineered strains (YGLY12903, YGLY27890), and selected for temperature-resistant mutants. These glyco-engineered strains are able to produce glycoproteins comprising sialylated N-glycans having an oligosaccharide structure selected from the group consisting of NANA₍₁₋₄₎Gal₍₁₋₄₎Man₃GlcNAc₂.

The geneology for strain YGLY12903 is as follows:

[ura5Δ::ScSUC2 och1Δ::lacZ bmt2Δ::lacZ/KlMNN2-2
mnn4L1Δ::lacZ/MmSLC35A3 pno1Δ mnn4Δ::lacZ
ADE1::lacZ/NA10/MmSLC35A3/FB8
his1Δ::lacZ/ScGAL10/XB33/DmUGT
arg1Δ::HIS1/KD53/TC54
bmt4Δ::lacZ bmt1Δ::lacZ bmt3Δ::lacZ

### TRP2::ARG1/MmCST/HsGNE/HsCSS/HsSPS/MmST6-33

ste13Δ::lacZ-URA5-lacZ/TrMDS1 dap2Δ::Nat^{R}

### TRP5::Hyg^{R}MmCST/HsGNE/HsCSS/HsSPS/MmST6-33]

The geneology for strain YGLY27890 is as follows:

[ura5Δ::ScSUC2 och1Δ::lacZ bmt2Δ::lacZ/K1MNN2-2
mnn4L1Δ::lacZ/MmSLC35A3 pno1Δ mnn4Δ::lacZ
ADE1::lacZ/NA10/MmSLC35A3/FB8
his1Δ:: lacZ/ScGAL10/XB33/DmUGT
arg1Δ::HIS1/KD53/TC54
bmt4Δ::lacZ bmt1Δ::lacZ bmt3Δ::lacZ

### TRP2::ARG1/MmCST/HsGNE/HsCSS/HsSPS/MmST6-33

ste13Δ::lacZ-URA5-lacZ/TrMDS1 dap2Δ::Nat^{R}

### TRP5::Hyg^{R}MmCST/HsGNE/HsCSS/HsSPS/MmST6-33

vps10-1::AOX1p_LmSTT3-URA5
TRP1::AOX1p_hFc-ZeoR]

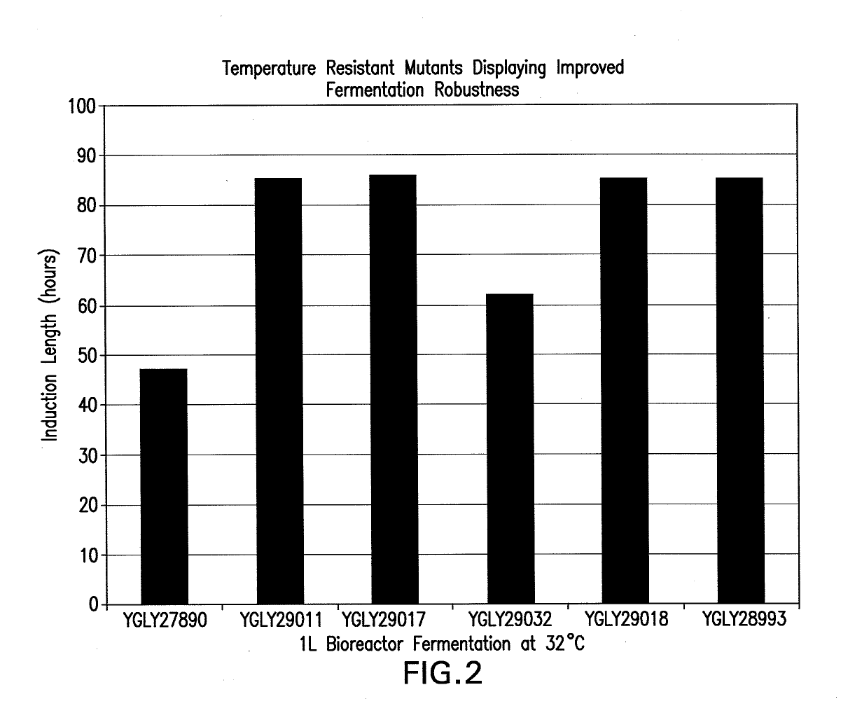

After confirming their temperature-resistant phenotypes, these mutants were fermented using standard MeOH fed-batch runs in 1 L DasGip Bioreactors. After an extensive fermentation screening campaign, we identified 4 mutants displaying much enhanced cell robustness during the fermentation process. As shown in FIG. 2, the fermentation process for the non-mutagenized control strain had to be terminated, due to excessive cell lysis, at approximately 48 hours of induction at 32° C. In contrast, the mutants (YGLY28993, YGLY29011, YGLY29017, and YGLY29032) all displayed significantly improved fermentation robustness. YGLY29032 was able to ferment more than 60 hours; YGLY28993, YGLY29011, and YGLY29017 all lasted for more than 80 hours induction at 32° C.

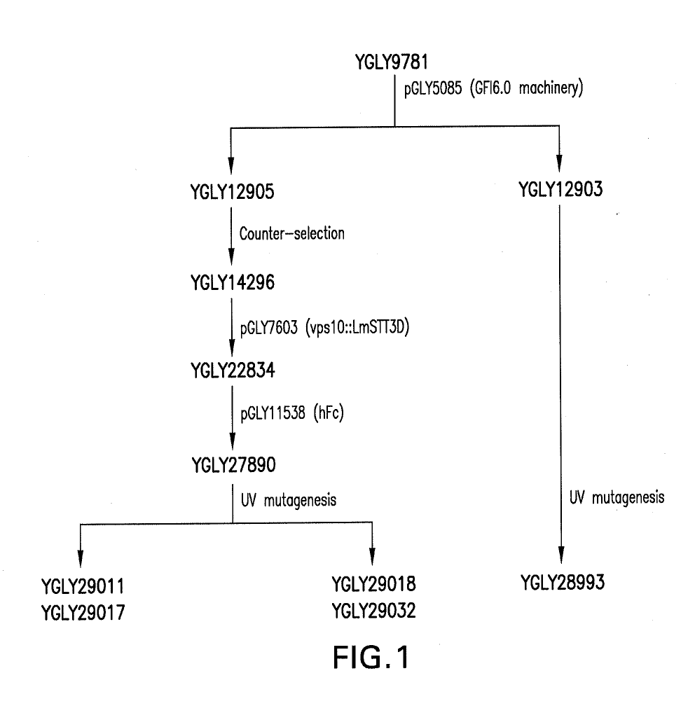

A representation of the strain lineages used in the experiments described herein is shown in FIG. 1.

### Example 2

### Genome Sequencing to Identify the Causative Mutation(s) Responsible for the Enhanced Thermal-Tolerance and Fermentation Robustness

To uncover the mutations responsible for this increased thermal tolerance and fermentation robustness, we performed genome-sequencing for 4 independently isolated mutants, (YGLY28993, YGLY29011, YGLY29017, and YGLY29032), as well as two un-mutagenized empty host strains YGLY22812 and YGLY22835. After genome-wide comparisons between the mutants and the un-mutagenized strains, we identified between 1 to 10 non-synonymous nucleotide variations (indicated by a “+” in Table 1) in each of these 4 mutants. One mutant, YGLY29011, contained a single mutation within a gene, Pp05g01920, which shows a high-level of sequence homology to the GRR1 gene of Saccharomyces cerevisiae. Distinct mutations in the same PpGRR1 gene were also identified in YGLY28993, YGLY29017, and YGLY29032.

TABLE 1

| | | | | | | | | | | ref- | read- |
|---|---|---|---|---|---|---|---|---|---|---|---|
| Chromosome | yGLY28993 | yGLY29011 | yGLY29032 | yGLY29017 | ref | read | gene-id | ref | read | a.a | a.a |
| chr1 | + | − | − | − | T | C | Pp01g07560.1 | GAA | GGA | E | G |
| chr1 | − | − | − | + | C | T | Pp01g08950 | TCT | TTT | S | F |
| chr1 | + | − | − | − | C | T | Pp01g12440 | TCT | TTT | S | F |
| chr2 | + | − | − | − | A | G | Pp02g00990 | AAT | GAT | N | D |
| chr2 | + | − | − | − | T | C | Pp02g01670 | TCC | CCC | S | P |
| chr2 | + | − | − | − | T | A | Pp02g06760 | TTA | TCA | L | S |
| chr3 | + | − | − | − | A | T | Pp03g00340 | GAA | GTA | E | V |
| chr3 | + | − | − | − | G | A | Pp03g06360 | TGG | TTT | W | F |
| chr3 | + | − | − | − | T | C | Pp03g06600 | GAA | CAA | E | Q |
| chr3 | − | − | + | − | A | G | Pp03g09410 | AAG | GAG | K | E |
| chr4 | + | − | − | − | G | A | Pp05g01920 | CGT | TGT | R | C |
| chr4 | − | + | − | − | G | A | Pp05g01920 | TCA | TTA | S | L |
| chr4 | − | − | + | − | A | G | Pp05g01920 | TTA | TCA | L | S |
| chr4 | − | − | − | + | A | G | Pp05g01920 | CTA | CCA | L | P |
| chr4 | + | − | − | − | C | T | Pp05g03540 | CCC | CTC | P | L |
| chr4 | − | − | − | + | C | T | Pp05g04250 | GTG | ATG | V | M |

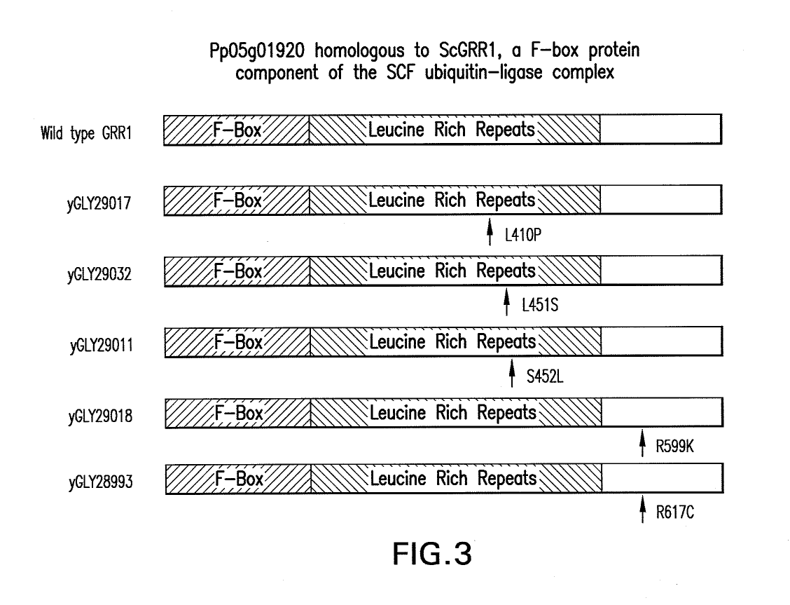

In Saccharomyces cerevisiae GRR1 is an F-box protein component of the SCF ubiquitin-ligase complex. F-box protein subunits are the substrate-binding component of the ubiquitin-ligase complex, and the specific region involved in substrate interactions for ScGRR1 is a leucine-rich repeat (LRR) domain. As illustrated in FIG. 3, three of the PpGRR1 mutations found from the temperature-resistant mutants were located within LRR domain, with 2 of them involving 2 leucine residues directly. The fourth PpGRR1 mutation is located shortly downstream of the LRR domain. The findings that four independently isolated temperature-resistant mutants contained different mis-sense mutation within the PpGRR1 gene strongly suggested that these GRR1 mutations were causative for the temperature-resistant and increased fermentation robustness phenotypes.

### Example 3

### Protein Productivity and N-Glycan Quality Assessments of the Temperature-Resistant Mutants

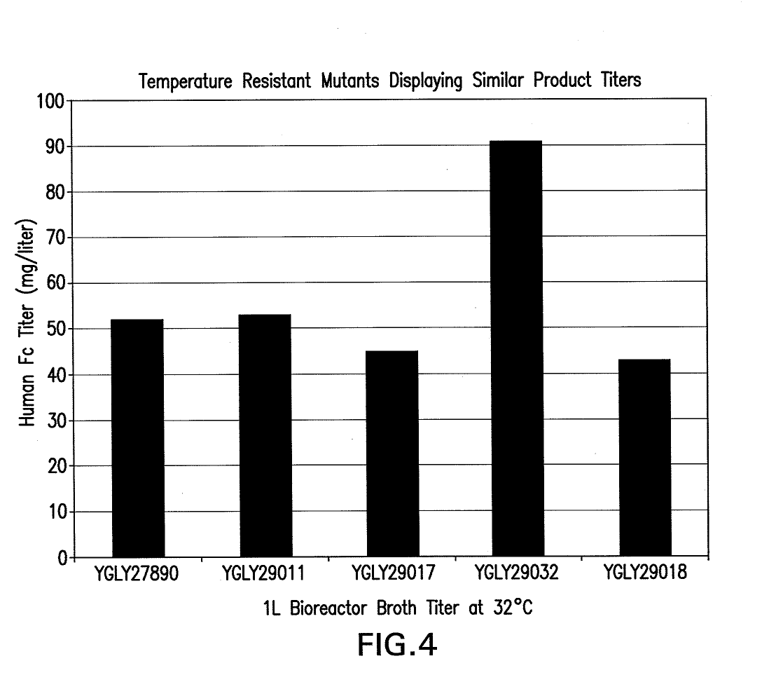

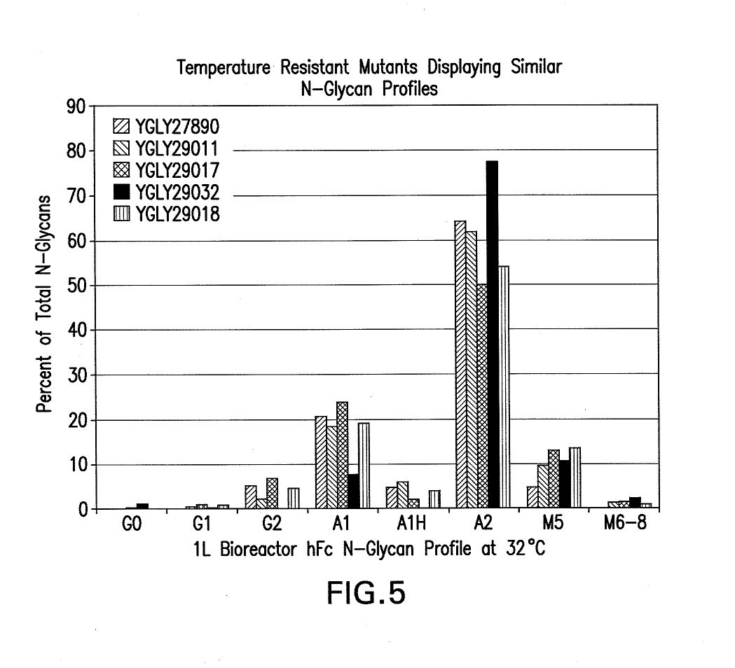

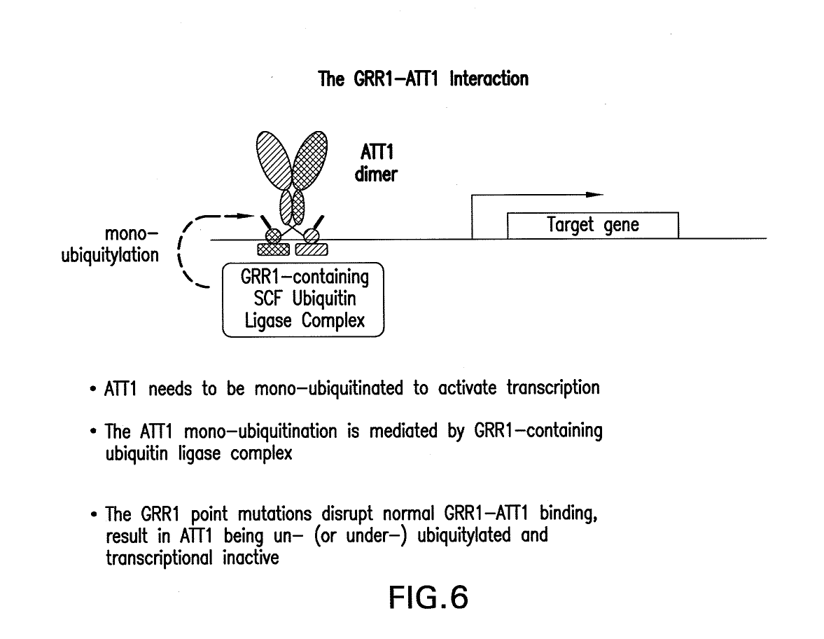

Three of the temperature-resistant mutants (YGLY29011, YGLY29017, and YGLY29032) were derived from YGLY27890, which expresses a human Fc fragment. To evaluate what impacts these temperature-resistant mutations had on Fc productivity and N-glycan quality, we purified the Fc fragments from the 32C 1 L bioreactors, quantified the broth titer (FIG. 4), and analyzed the N-glycan profiles (FIG. 5) of these three temperature-resistant mutants, as well as their un-mutagenized parent strain YGLY27890. Compared with the parental control, none of the mutants displayed a reduction in the product titers: in fact, YGLY29032 actually secreted approximately 80% more Fc product. Similarly, we did not observe any large alterations in the Fc N-glycan profiles (FIG. 5). Just like the control strain YGLY27890 (64% A2 and 21% A1), all three mutants were able to effectively modify their Fc N-glycans with high levels of terminal sialic acids, with A2 levels ranging from 50 to 77%, and A1 levels from 8 to 24%. Collectively, these results demonstrated that the UV-induced mutations acquired by YGLY29011, YGLY29017, and YGLY29032 did not negatively affect their capabilities for producing heterologously expressed human Fc fragment, nor did the mutations resulted in noticeable deteriorations in N-glycan quality.

### Example 4

### Confirmation of Phenotype by Directed Strain Engineering

Independent mutations in the same gene in each of the mutants strongly indicates that truncations of this GRR1 gene are responsible for the observed temperature-resistance and fermentation robustness phenotypes. To test this hypothesis, the endogenous GRR1 gene can be replaced in non-mutagenized Pichia strains with mutated versions corresponding to the mis-sense mutations observed in each mutant, and tested for an increase in both thermal-tolerance and fermentation robustness.

### GLOSSARY

OCH1: Alpha-1,6-mannosyltransferase
KlMNN2-2: K. lactis UDP-GlcNAc transporter
BMT1: Beta-mannose-transfer (beta-mannose elimination)
BMT2: Beta-mannose-transfer (beta-mannose elimination)
BMT3: Beta-mannose-transfer (beta-mannose elimination)
BMT4: Beta-mannose-transfer (beta-mannose elimination)
MNN4L1: MNN4-like 1 (charge elimination)
MmSLC35A3: Mouse homologue of UDP-GlcNAc transporter
PNO1: Phosphomannosylation of N-linked oligosaccharides (charge elimination)
MNN4: Mannosyltransferase (charge elimination)
ScGAL10: UDP-glucose 4-epimerase
XB33: Truncated HsGalT1 fused to ScKRE2 leader
DmUGT: UDP-Galactose transporter
KD53: Truncated DmMNSII fused to ScMNN2 leader
TC54: Truncated RnGNTII fused to ScMNN2 leader
NA10: Truncated HsGNTI fused to PpSEC12 leader
FB8: Truncated MmMNS1A fused to ScSEC12 leader
CiMNS1: Secreted Coccidioides immitis mannosidase I
LmSTT3D: Leishmania major oligosaccharyl transferase subunit D
ScSUC2: S. cerevisiae invertase
MmSLC35A3: Mouse orthologue of UDP-GlcNAc transporter
STE13 Golgi dipeptidyl aminopeptidase
DAP2 Vacuolar dipeptidyl aminopeptidase
ALG3 dolichol-P-Man dependent alpha(1-3) mannosyltransferase
POMGNT1 protein 0-mannose beta-1,2-N-acetylglucosaminyltransferase

Patents, patent applications, publications, product descriptions, and protocols are cited throughout this application, the disclosures of which are incorporated herein by reference in their entireties for all purposes. All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37 C.F.R. §1.57(b)(1), to relate to each and every individual publication, database entry (e.g. Genbank sequences or GeneID entries), patent application, or patent, each of which is clearly identified in compliance with 37 C.F.R. §1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The present invention is not to be limited in scope by the specific embodiments described herein; the embodiments specifically set forth herein are not necessarily intended to be exhaustive. Indeed, various modifications of the invention in addition to those described herein will become apparent to those skilled in the art from the foregoing description and the accompanying figures. Such modifications are intended to fall within the scope of the appended claims.

The foregoing written specification is considered to be sufficient to enable one skilled in the art to practice the invention. Various modifications of the invention in addition to those shown and described herein will become apparent to those skilled in the art from the foregoing description and fall within the scope of the appended claims.

TABLE 2
List of Sequences
and Brief Description

| | |
|---|---|
| SEQ ID | ATGCAGAGTAATTCGGAGAGAGACTCTTCGCCT |
| NO: 1 | AGTGACTCAAATAGCACCATTGAGTTGCAAAGA |
| PpGRR1 | TCCGAGAACGAATATGATCACCTAACTAATACG |
| wild type | ATAATGGAAGATCTGGGGCAAAAACTTAACCAC |
| open | TACAAGGAATCCCAGGACACGAGCTCCAGCCAT |
| reading | ATTTTACACTTACCTACTGAGGTTTTGCTACTC |
| frame | ATTTTATCATTTGTGACTTCGAAGACTGATCTT |
| | CTTAGTTTTATGTTGACATGTAGAAAGTTCGGA |
| | GACCTGGTTAGCGGTTTGCTCTGGTTCAGACCT |
| | GGTATTTCCAATGCATACGTCTATAAAGAAATG |
| | ATCAGAATAATGAGAATACCTCCAGAGAAGACA |
| | TTTTGGGACTACAAAAAGTTTATCAGAAGATTG |
| | AATCTGTCCCTGGTTTCTAACTTGGTTGAGGAT |
| | GAGTTCCTATATGCATTCAGTGGTTGCCCCAAC |
| | CTGGAAAGGATCACATTAGTGAATTGCAGTAAA |
| | GTTACTGCTGATTCTGTGGCGACAATATTGAAG |
| | GATGCATCCAACCTTCAGTCTATTGACCTTACA |
| | GGAGTTGTGAATATCACAGATGGAGTCTACTAC |
| | AGTTTAGCACGCCACTGTAAGAAACTGCAGGGT |
| | CTATATGCCCCAGGTTCTATGGCTGTTTCCAAG |
| | AACGCAGTGTACACTCTCATATCCAATTGCCCA |
| | ATGCTGAAAAGAATCAAACTGAGTGAATGTGTG |
| | GGAGTAGACGATGAGATTGTTGTGAAATTGGTG |
| | AGAGAATGTAAAAATCTCGTCGAATTAGACCTT |
| | CATGGGTGTATCAGAGTTACCGATTATGCTCTA |
| | GTTGTGCTTTTTGAAGAATTGGAATATTTGAGA |
| | GAGTTCAAAATCTCAATGAATGATCATATAACA |
| | GAGAGATGCTTCCTTGGGCTACCAAACGAGCCC |
| | TACTTGGATAAGCTTAGGATAATTGATTTCACT |
| | AGTTGCAGCAATGTTAACGACAAACTTGTCATC |
| | AAGTTAGTTCAATTAGCACCCAAGTTGAGGCAT |
| | ATTGTATTGTCTAAGTGTACCAAAATAACGGAC |
| | TCGTCCTTGAGAGCCTTAGCAACTTTGGGCAAG |
| | TGCTTGCACTACTTGCATCTGGGACATTGTATT |
| | AACATAACAGATTTTGGAGTCTGTCATCTGCTT |
| | AGAAATTGTCATCGACTTCAGTATGTCGATCTT |
| | GCATGCTGTCAAGAGCTGACCAATGACACCTTG |
| | TTTGAACTATCTCAGTTACCAAGATTGAGAAGA |
| | ATTGGCTTGGTGAAATGTCACAATATAACCGAT |
| | CATGGCATTTTGTATCTAGCAAATAACCGGAGA |
| | TCGCCAGACGATACTTTAGAAAGAGTACATTTA |
| | TCATATTGTACACAGATTAGCATATTTCCTATC |
| | TACAAGTTACTAATGGCGTGTCGCAGACTGACA |
| | CACCTATCATTAACAGGTATCAGAGACTTCTTG |
| | AGAAGTGATATTACAAGATTTTGCCGAGATCCT |
| | CCCAATGACTTTACTCAATCTCAAAGAGATATG |
| | TTTTGTGTTTTCAGTGGTGACGGGGTCCGGAAG |
| | CTTCGAGATCACCTTTCTAGTCTCTACCACCAA |
| | CAGCAACAAATTAACAGATACGTTAACTCTCAA |
| | AATATAGGAAACTTAAGGGATGACGGAGAGACT |
| | TTGAATGAGATCTTCCAGTATATTGCAAATCCA |
| | GCCACCCCTGGACAACTACCCCCAAGGGTCCAA |
| | GAACTCGTTGAAGCAAGACGGAGAAACAGAAAT |
| | CAAGAACGCATAATGACTAACACTGTTAACTTT |
| | CCTGAACAAATTAGAAGGTTGTCTCTCCTGCCT |
| | CCGGAACAACAACAGGCTTTGCCACAACCAATT |
| | CGTCAACTGATTGCCCAAGCCACTGCATCTCCG |
| | CTATCTTTTCCTTTACAAGATCAGGAGCCACAA |
| | CAGCAGCAACAACAAGAAAGGGGTCTTGGCATT |
| | CCGCCAGTTGATAACTTCAGTCCGGTAGTTGAT |
| | GAAGGGCAAGAATATGATGAAGACCAAGAGATG |
| | GAA |
| SEQ ID | ATGCAGAGTAATTCGGAGAGAGACTCTTCGCCT |
| NO: 2 | AGTGACTCAAATAGCACCATTGAGTTGCAAAGA |
| PpGRR1 | TCCGAGAACGAATATGATCACCTAACTAATACG |
| (L410P) | ATAATGGAAGATCTGGGGCAAAAACTTAACCAC |
| mutant | TACAAGGAATCCCAGGACACGAGCTCCAGCCAT |
| ORF | ATTTTACACTTACCTACTGAGGTTTTGCTACTC |
| | ATTTTATCATTTGTGACTTCGAAGACTGATCTT |
| | CTTAGTTTTATGTTGACATGTAGAAAGTTCGGA |
| | GACCTGGTTAGCGGTTTGCTCTGGTTCAGACCT |
| | GGTATTTCCAATGCATACGTCTATAAAGAAATG |
| | ATCAGAATAATGAGAATACCTCCAGAGAAGACA |
| | TTTTGGGACTACAAAAAGTTTATCAGAAGATTG |
| | AATCTGTCCCTGGTTTCTAACTTGGTTGAGGAT |
| | GAGTTCCTATATGCATTCAGTGGTTGCCCCAAC |
| | CTGGAAAGGATCACATTAGTGAATTGCAGTAAA |
| | GTTACTGCTGATTCTGTGGCGACAATATTGAAG |
| | GATGCATCCAACCTTCAGTCTATTGACCTTACA |
| | GGAGTTGTGAATATCACAGATGGAGTCTACTAC |
| | AGTTTAGCACGCCACTGTAAGAAACTGCAGGGT |
| | CTATATGCCCCAGGTTCTATGGCTGTTTCCAAG |
| | AACGCAGTGTACACTCTCATATCCAATTGCCCA |
| | ATGCTGAAAAGAATCAAACTGAGTGAATGTGTG |
| | GGAGTAGACGATGAGATTGTTGTGAAATTGGTG |
| | AGAGAATGTAAAAATCTCGTCGAATTAGACCTT |
| | CATGGGTGTATCAGAGTTACCGATTATGCTCTA |
| | GTTGTGCTTTTTGAAGAATTGGAATATTTGAGA |
| | GAGTTCAAAATCTCAATGAATGATCATATAACA |
| | GAGAGATGCTTCCTTGGGCTACCAAACGAGCCC |
| | TACTTGGATAAGCTTAGGATAATTGATTTCACT |
| | AGTTGCAGCAATGTTAACGACAAACTTGTCATC |
| | AAGTTAGTTCAATTAGCACCCAAGTTGAGGCAT |
| | ATTGTATTGTCTAAGTGTACCAAAATAACGGAC |
| | TCGTCCTTGAGAGCCTTAGCAACTTTGGGCAAG |
| | TGCTTGCACTACTTGCATCTGGGACATTGTATT |
| | AACATAACAGATTTTGGAGTCTGTCATCTGCTT |
| | AGAAATTGTCATCGACTTCAGTATGTCGATCTT |
| | GCATGCTGTCAAGAGCTGACCAATGACACCTTG |
| | TTTGAACCATCTCAGTTACCAAGATTGAGAAGA |
| | ATTGGCTTGGTGAAATGTCACAATATAACCGAT |
| | CATGGCATTTTGTATCTAGCAAATAACCGGAGA |
| | TCGCCAGACGATACTTTAGAAAGAGTACATTTA |
| | TCATATTGTACACAGATTAGCATATTTCCTATC |
| | TACAAGTTACTAATGGCGTGTCGCAGACTGACA |
| | CACCTATCATTAACAGGTATCAGAGACTTCTTG |
| | AGAAGTGATATTACAAGATTTTGCCGAGATCCT |
| | CCCAATGACTTTACTCAATCTCAAAGAGATATG |
| | TTTTGTGTTTTCAGTGGTGACGGGGTCCGGAAG |
| | CTTCGAGATCACCTTTCTAGTCTCTACCACCAA |
| | CAGCAACAAATTAACAGATACGTTAACTCTCAA |
| | AATATAGGAAACTTAAGGGATGACGGAGAGACT |
| | TTGAATGAGATCTTCCAGTATATTGCAAATCCA |
| | GCCACCCCTGGACAACTACCCCCAAGGGTCCAA |
| | GAACTCGTTGAAGCAAGACGGAGAAACAGAAAT |
| | CAAGAACGCATAATGACTAACACTGTTAACTTT |
| | CCTGAACAAATTAGAAGGTTGTCTCTCCTGCCT |
| | CCGGAACAACAACAGGCTTTGCCACAACCAATT |
| | CGTCAACTGATTGCCCAAGCCACTGCATCTCCG |
| | CTATCTTTTCCTTTACAAGATCAGGAGCCACAA |
| | CAGCAGCAACAACAAGAAAGGGGTCTTGGCATT |
| | CCGCCAGTTGATAACTTCAGTCCGGTAGTTGAT |
| | GAAGGGCAAGAATATGATGAAGACCAAGAGATG |
| | GAA |
| SEQ ID | ATGCAGAGTAATTCGGAGAGAGACTCTTCGCCT |
| NO: 3 | AGTGACTCAAATAGCACCATTGAGTTGCAAAGA |
| PpGRR1 | TCCGAGAACGAATATGATCACCTAACTAATACG |
| (L451S) | ATAATGGAAGATCTGGGGCAAAAACTTAACCAC |
| mutant | TACAAGGAATCCCAGGACACGAGCTCCAGCCAT |
| ORF | ATTTTACACTTACCTACTGAGGTTTTGCTACTC |
| | ATTTTATCATTTGTGACTTCGAAGACTGATCTT |
| | CTTAGTTTTATGTTGACATGTAGAAAGTTCGGA |
| | GACCTGGTTAGCGGTTTGCTCTGGTTCAGACCT |
| | GGTATTTCCAATGCATACGTCTATAAAGAAATG |
| | ATCAGAATAATGAGAATACCTCCAGAGAAGACA |
| | TTTTGGGACTACAAAAAGTTTATCAGAAGATTG |
| | AATCTGTCCCTGGTTTCTAACTTGGTTGAGGAT |
| | GAGTTCCTATATGCATTCAGTGGTTGCCCCAAC |
| | CTGGAAAGGATCACATTAGTGAATTGCAGTAAA |
| | GTTACTGCTGATTCTGTGGCGACAATATTGAAG |
| | GATGCATCCAACCTTCAGTCTATTGACCTTACA |
| | GGAGTTGTGAATATCACAGATGGAGTCTACTAC |
| | AGTTTAGCACGCCACTGTAAGAAACTGCAGGGT |
| | CTATATGCCCCAGGTTCTATGGCTGTTTCCAAG |
| | AACGCAGTGTACACTCTCATATCCAATTGCCCA |
| | ATGCTGAAAAGAATCAAACTGAGTGAATGTGTG |
| | GGAGTAGACGATGAGATTGTTGTGAAATTGGTG |
| | AGAGAATGTAAAAATCTCGTCGAATTAGACCTT |
| | CATGGGTGTATCAGAGTTACCGATTATGCTCTA |
| | GTTGTGCTTTTTGAAGAATTGGAATATTTGAGA |
| | GAGTTCAAAATCTCAATGAATGATCATATAACA |
| | GAGAGATGCTTCCTTGGGCTACCAAACGAGCCC |
| | TACTTGGATAAGCTTAGGATAATTGATTTCACT |
| | AGTTGCAGCAATGTTAACGACAAACTTGTCATC |
| | AAGTTAGTTCAATTAGCACCCAAGTTGAGGCAT |
| | ATTGTATTGTCTAAGTGTACCAAAATAACGGAC |
| | TCGTCCTTGAGAGCCTTAGCAACTTTGGGCAAG |
| | TGCTTGCACTACTTGCATCTGGGACATTGTATT |
| | AACATAACAGATTTTGGAGTCTGTCATCTGCTT |
| | AGAAATTGTCATCGACTTCAGTATGTCGATCTT |
| | GCATGCTGTCAAGAGCTGACCAATGACACCTTG |
| | TTTGAACTATCTCAGTTACCAAGATTGAGAAGA |
| | ATTGGCTTGGTGAAATGTCACAATATAACCGAT |
| | CATGGCATTTTGTATCTAGCAAATAACCGGAGA |
| | TCGCCAGACGATACTTTAGAAAGAGTACATTCA |
| | TCATATTGTACACAGATTAGCATATTTCCTATC |
| | TACAAGTTACTAATGGCGTGTCGCAGACTGACA |
| | CACCTATCATTAACAGGTATCAGAGACTTCTTG |
| | AGAAGTGATATTACAAGATTTTGCCGAGATCCT |
| | CCCAATGACTTTACTCAATCTCAAAGAGATATG |
| | TTTTGTGTTTTCAGTGGTGACGGGGTCCGGAAG |
| | CTTCGAGATCACCTTTCTAGTCTCTACCACCAA |
| | CAGCAACAAATTAACAGATACGTTAACTCTCAA |
| | AATATAGGAAACTTAAGGGATGACGGAGAGACT |
| | TTGAATGAGATCTTCCAGTATATTGCAAATCCA |
| | GCCACCCCTGGACAACTACCCCCAAGGGTCCAA |
| | GAACTCGTTGAAGCAAGACGGAGAAACAGAAAT |
| | CAAGAACGCATAATGACTAACACTGTTAACTTT |
| | CCTGAACAAATTAGAAGGTTGTCTCTCCTGCCT |
| | CCGGAACAACAACAGGCTTTGCCACAACCAATT |
| | CGTCAACTGATTGCCCAAGCCACTGCATCTCCG |
| | CTATCTTTTCCTTTACAAGATCAGGAGCCACAA |
| | CAGCAGCAACAACAAGAAAGGGGTCTTGGCATT |
| | CCGCCAGTTGATAACTTCAGTCCGGTAGTTGAT |
| | GAAGGGCAAGAATATGATGAAGACCAAGAGATG |
| | GAA |
| SEQ ID | ATGCAGAGTAATTCGGAGAGAGACTCTTCGCCT |
| NO: 4 | AGTGACTCAAATAGCACCATTGAGTTGCAAAGA |
| PpGRR1 | TCCGAGAACGAATATGATCACCTAACTAATACG |
| (S452L) | ATAATGGAAGATCTGGGGCAAAAACTTAACCAC |
| mutant | TACAAGGAATCCCAGGACACGAGCTCCAGCCAT |
| ORF | ATTTTACACTTACCTACTGAGGTTTTGCTACTC |
| | ATTTTATCATTTGTGACTTCGAAGACTGATCTT |
| | CTTAGTTTTATGTTGACATGTAGAAAGTTCGGA |
| | GACCTGGTTAGCGGTTTGCTCTGGTTCAGACCT |
| | GGTATTTCCAATGCATACGTCTATAAAGAAATG |
| | ATCAGAATAATGAGAATACCTCCAGAGAAGACA |
| | TTTTGGGACTACAAAAAGTTTATCAGAAGATTG |
| | AATCTGTCCCTGGTTTCTAACTTGGTTGAGGAT |
| | GAGTTCCTATATGCATTCAGTGGTTGCCCCAAC |
| | CTGGAAAGGATCACATTAGTGAATTGCAGTAAA |
| | GTTACTGCTGATTCTGTGGCGACAATATTGAAG |
| | GATGCATCCAACCTTCAGTCTATTGACCTTACA |
| | GGAGTTGTGAATATCACAGATGGAGTCTACTAC |
| | AGTTTAGCACGCCACTGTAAGAAACTGCAGGGT |
| | CTATATGCCCCAGGTTCTATGGCTGTTTCCAAG |
| | AACGCAGTGTACACTCTCATATCCAATTGCCCA |
| | ATGCTGAAAAGAATCAAACTGAGTGAATGTGTG |
| | GGAGTAGACGATGAGATTGTTGTGAAATTGGTG |
| | AGAGAATGTAAAAATCTCGTCGAATTAGACCTT |
| | CATGGGTGTATCAGAGTTACCGATTATGCTCTA |
| | GTTGTGCTTTTTGAAGAATTGGAATATTTGAGA |
| | GAGTTCAAAATCTCAATGAATGATCATATAACA |
| | GAGAGATGCTTCCTTGGGCTACCAAACGAGCCC |
| | TACTTGGATAAGCTTAGGATAATTGATTTCACT |
| | AGTTGCAGCAATGTTAACGACAAACTTGTCATC |
| | AAGTTAGTTCAATTAGCACCCAAGTTGAGGCAT |
| | ATTGTATTGTCTAAGTGTACCAAAATAACGGAC |
| | TCGTCCTTGAGAGCCTTAGCAACTTTGGGCAAG |
| | TGCTTGCACTACTTGCATCTGGGACATTGTATT |
| | AACATAACAGATTTTGGAGTCTGTCATCTGCTT |
| | AGAAATTGTCATCGACTTCAGTATGTCGATCTT |
| | GCATGCTGTCAAGAGCTGACCAATGACACCTTG |
| | TTTGAACTATCTCAGTTACCAAGATTGAGAAGA |
| | ATTGGCTTGGTGAAATGTCACAATATAACCGAT |
| | CATGGCATTTTGTATCTAGCAAATAACCGGAGA |
| | TCGCCAGACGATACTTTAGAAAGAGTACATTTA |
| | TTATATTGTACACAGATTAGCATATTTCCTATC |
| | TACAAGTTACTAATGGCGTGTCGCAGACTGACA |
| | CACCTATCATTAACAGGTATCAGAGACTTCTTG |
| | AGAAGTGATATTACAAGATTTTGCCGAGATCCT |
| | CCCAATGACTTTACTCAATCTCAAAGAGATATG |
| | TTTTGTGTTTTCAGTGGTGACGGGGTCCGGAAG |
| | CTTCGAGATCACCTTTCTAGTCTCTACCACCAA |
| | CAGCAACAAATTAACAGATACGTTAACTCTCAA |
| | AATATAGGAAACTTAAGGGATGACGGAGAGACT |
| | TTGAATGAGATCTTCCAGTATATTGCAAATCCA |
| | GCCACCCCTGGACAACTACCCCCAAGGGTCCAA |
| | GAACTCGTTGAAGCAAGACGGAGAAACAGAAAT |
| | CAAGAACGCATAATGACTAACACTGTTAACTTT |
| | CCTGAACAAATTAGAAGGTTGTCTCTCCTGCCT |
| | CCGGAACAACAACAGGCTTTGCCACAACCAATT |
| | CGTCAACTGATTGCCCAAGCCACTGCATCTCCG |
| | CTATCTTTTCCTTTACAAGATCAGGAGCCACAA |
| | CAGCAGCAACAACAAGAAAGGGGTCTTGGCATT |
| | CCGCCAGTTGATAACTTCAGTCCGGTAGTTGAT |
| | GAAGGGCAAGAATATGATGAAGACCAAGAGATG |
| | GAA |
| SEQ ID | ATGCAGAGTAATTCGGAGAGAGACTCTTCGCCT |
| NO: 5 | AGTGACTCAAATAGCACCATTGAGTTGCAAAGA |
| PpGRR1 | TCCGAGAACGAATATGATCACCTAACTAATACG |
| (R617C) | ATAATGGAAGATCTGGGGCAAAAACTTAACCAC |
| mutant | TACAAGGAATCCCAGGACACGAGCTCCAGCCAT |
| ORF | ATTTTACACTTACCTACTGAGGTTTTGCTACTC |
| | ATTTTATCATTTGTGACTTCGAAGACTGATCTT |
| | CTTAGTTTTATGTTGACATGTAGAAAGTTCGGA |
| | GACCTGGTTAGCGGTTTGCTCTGGTTCAGACCT |
| | GGTATTTCCAATGCATACGTCTATAAAGAAATG |
| | ATCAGAATAATGAGAATACCTCCAGAGAAGACA |
| | TTTTGGGACTACAAAAAGTTTATCAGAAGATTG |
| | AATCTGTCCCTGGTTTCTAACTTGGTTGAGGAT |
| | GAGTTCCTATATGCATTCAGTGGTTGCCCCAAC |
| | CTGGAAAGGATCACATTAGTGAATTGCAGTAAA |
| | GTTACTGCTGATTCTGTGGCGACAATATTGAAG |
| | GATGCATCCAACCTTCAGTCTATTGACCTTACA |
| | GGAGTTGTGAATATCACAGATGGAGTCTACTAC |
| | AGTTTAGCACGCCACTGTAAGAAACTGCAGGGT |
| | CTATATGCCCCAGGTTCTATGGCTGTTTCCAAG |
| | AACGCAGTGTACACTCTCATATCCAATTGCCCA |
| | ATGCTGAAAAGAATCAAACTGAGTGAATGTGTG |
| | GGAGTAGACGATGAGATTGTTGTGAAATTGGTG |
| | AGAGAATGTAAAAATCTCGTCGAATTAGACCTT |
| | CATGGGTGTATCAGAGTTACCGATTATGCTCTA |
| | GTTGTGCTTTTTGAAGAATTGGAATATTTGAGA |
| | GAGTTCAAAATCTCAATGAATGATCATATAACA |
| | GAGAGATGCTTCCTTGGGCTACCAAACGAGCCC |
| | TACTTGGATAAGCTTAGGATAATTGATTTCACT |
| | AGTTGCAGCAATGTTAACGACAAACTTGTCATC |
| | AAGTTAGTTCAATTAGCACCCAAGTTGAGGCAT |
| | ATTGTATTGTCTAAGTGTACCAAAATAACGGAC |
| | TCGTCCTTGAGAGCCTTAGCAACTTTGGGCAAG |
| | TGCTTGCACTACTTGCATCTGGGACATTGTATT |
| | AACATAACAGATTTTGGAGTCTGTCATCTGCTT |
| | AGAAATTGTCATCGACTTCAGTATGTCGATCTT |
| | GCATGCTGTCAAGAGCTGACCAATGACACCTTG |
| | TTTGAACTATCTCAGTTACCAAGATTGAGAAGA |
| | ATTGGCTTGGTGAAATGTCACAATATAACCGAT |
| | CATGGCATTTTGTATCTAGCAAATAACCGGAGA |
| | TCGCCAGACGATACTTTAGAAAGAGTACATTTA |
| | TCATATTGTACACAGATTAGCATATTTCCTATC |
| | TACAAGTTACTAATGGCGTGTCGCAGACTGACA |
| | CACCTATCATTAACAGGTATCAGAGACTTCTTG |
| | AGAAGTGATATTACAAGATTTTGCCGAGATCCT |
| | CCCAATGACTTTACTCAATCTCAAAGAGATATG |
| | TTTTGTGTTTTCAGTGGTGACGGGGTCCGGAAG |
| | CTTCGAGATCACCTTTCTAGTCTCTACCACCAA |
| | CAGCAACAAATTAACAGATACGTTAACTCTCAA |
| | AATATAGGAAACTTAAGGGATGACGGAGAGACT |
| | TTGAATGAGATCTTCCAGTATATTGCAAATCCA |
| | GCCACCCCTGGACAACTACCCCCAAGGGTCCAA |
| | GAACTCGTTGAAGCAAGACGGAGAAACAGAAAT |
| | CAAGAACGCATAATGACTAACACTGTTAACTTT |
| | CCTGAACAAATTAGAAGGTTGTCTCTCCTGCCT |
| | CCGGAACAACAACAGGCTTTGCCACAACCAATT |
| | TGTCAACTGATTGCCCAAGCCACTGCATCTCCG |
| | CTATCTTTTCCTTTACAAGATCAGGAGCCACAA |
| | CAGCAGCAACAACAAGAAAGGGGTCTTGGCATT |
| | CCGCCAGTTGATAACTTCAGTCCGGTAGTTGAT |
| | GAAGGGCAAGAATATGATGAAGACCAAGAGATG |
| | GAA |
| SEQ ID | MQSNSERDSSPSDSNSTIELQRSENEYDHLTNT |
| NO: 6 | IMEDLGQKLNHYKESQDTSSSHILHLPTEVLLL |
| PpGRR1 | ILSFVTSKTDLLSFMLTCRKFGDLVSGLLWFRP |
| wild type | GISNAYVYKEMIRIMRIPPEKTFWDYKKFIRRL |
| amino | NLSLVSNLVEDEFLYAFSGCPNLERITLVNCSK |
| acid | VTADSVATILKDASNLQSIDLTGVVNITDGVYY |
| sequence | SLARHCKKLQGLYAPGSMAVSKNAVYTLISNCP |
| | MLKRIKLSECVGVDDEIVVKLVRECKNLVELDL |
| | HGCIRVTDYALVVLFEELEYLREFKISMNDHIT |
| | ERCFLGLPNEPYLDKLRIIDFTSCSNVNDKLVI |
| | KLVQLAPKLRHIVLSKCTKITDSSLRALATLGK |
| | CLHYLHLGHCINITDFGVCHLLRNCHRLQYVDL |
| | ACCQELTNDTLFELSQLPRLRRIGLVKCHNITD |
| | HGILYLANNRRSPDDTLERVHLSYCTQISIFPI |
| | YKLLMACRRLTHLSLTGIRDFLRSDITRFCRDP |
| | PNDFTQSQRDMFCVFSGDGVRKLRDHLSSLYHQ |
| | QQQINRYVNSQNIGNLRDDGETLNEIFQYIANP |
| | ATPGQLPPRVQELVEARRRNRNQERIMTNTVNF |
| | PEQIRRLSLLPPEQQQALPQPIRQLIAQATASP |
| | LSFPLQDQEPQQQQQQERGLGIPPVDNFSPVVD |
| | EGQEYDEDQEME |
| SEQ ID | MQSNSERDSSPSDSNSTIELQRSENEYDHLTNT |
| NO: 7 | IMEDLGQKLNHYKESQDTSSSHILHLPTEVLLL |
| PpGRR1 | ILSFVTSKTDLLSFMLTCRKFGDLVSGLLWFRP |
| (L410P) | GISNAYVYKEMIRIMRIPPEKTFWDYKKFIRRL |
| mutant | NLSLVSNLVEDEFLYAFSGCPNLERITLVNCSK |
| | VTADSVATILKDASNLQSIDLTGVVNITDGVYY |
| | SLARHCKKLQGLYAPGSMAVSKNAVYTLISNCP |
| | MLKRIKLSECVGVDDEIVVKLVRECKNLVELDL |
| | HGCIRVTDYALVVLFEELEYLREFKISMNDHIT |
| | ERCFLGLPNEPYLDKLRIIDFTSCSNVNDKLVI |
| | KLVQLAPKLRHIVLSKCTKITDSSLRALATLGK |
| | CLHYLHLGHCINITDFGVCHLLRNCHRLQYVDL |
| | ACCQELTNDTLFEPSQLPRLRRIGLVKCHNITD |
| | HGILYLANNRRSPDDTLERVHLSYCTQISIFPI |
| | YKLLMACRRLTHLSLTGIRDFLRSDITRFCRDP |
| | PNDFTQSQRDMFCVFSGDGVRKLRDHLSSLYHQ |
| | QQQINRYVNSQNIGNLRDDGETLNEIFQYIANP |
| | ATPGQLPPRVQELVEARRRNRNQERIMTNTVNF |
| | PEQIRRLSLLPPEQQQALPQPIRQLIAQATASP |
| | LSFPLQDQEPQQQQQQERGLGIPPVDNFSPVVD |
| | EGQEYDEDQEME |
| SEQ ID | MQSNSERDSSPSDSNSTIELQRSENEYDHLTNT |
| NO: 8 | IMEDLGQKLNHYKESQDTSSSHILHLPTEVLLL |
| PpGRR1 | ILSFVTSKTDLLSFMLTCRKFGDLVSGLLWFRP |
| (L451S) | GISNAYVYKEMIRIMRIPPEKTFWDYKKFIRRL |
| mutant | NLSLVSNLVEDEFLYAFSGCPNLERITLVNCSK |
| | VTADSVATILKDASNLQSIDLTGVVNITDGVYY |
| | SLARHCKKLQGLYAPGSMAVSKNAVYTLISNCP |
| | MLKRIKLSECVGVDDEIVVKLVRECKNLVELDL |
| | HGCIRVTDYALVVLFEELEYLREFKISMNDHIT |
| | ERCFLGLPNEPYLDKLRIIDFTSCSNVNDKLVI |
| | KLVQLAPKLRHIVLSKCTKITDSSLRALATLGK |
| | CLHYLHLGHCINITDFGVCHLLRNCHRLQYVDL |
| | ACCQELTNDTLFELSQLPRLRRIGLVKCHNITD |
| | HGILYLANNRRSPDDTLERVHSSYCTQISIFPI |
| | YKLLMACRRLTHLSLTGIRDFLRSDITRFCRDP |
| | PNDFTQSQRDMFCVFSGDGVRKLRDHLSSLYHQ |
| | QQQINRYVNSQNIGNLRDDGETLNEIFQYIANP |
| | ATPGQLPPRVQELVEARRRNRNQERIMTNTVNF |
| | PEQIRRLSLLPPEQQQALPQPIRQLIAQATASP |
| | LSFPLQDQEPQQQQQQERGLGIPPVDNFSPVVD |
| | EGQEYDEDQEME |
| SEQ ID | MQSNSERDSSPSDSNSTIELQRSENEYDHLTNT |
| NO: 9 | IMEDLGQKLNHYKESQDTSSSHILHLPTEVLLL |
| PpGRR1 | ILSFVTSKTDLLSFMLTCRKFGDLVSGLLWFRP |
| (S452L) | GISNAYVYKEMIRIMRIPPEKTFWDYKKFIRRL |
| mutant | NLSLVSNLVEDEFLYAFSGCPNLERITLVNCSK |
| | VTADSVATILKDASNLQSIDLTGVVNITDGVYY |
| | SLARHCKKLQGLYAPGSMAVSKNAVYTLISNCP |
| | MLKRIKLSECVGVDDEIVVKLVRECKNLVELDL |
| | HGCIRVTDYALVVLFEELEYLREFKISMNDHIT |
| | ERCFLGLPNEPYLDKLRIIDFTSCSNVNDKLVI |
| | KLVQLAPKLRHIVLSKCTKITDSSLRALATLGK |
| | CLHYLHLGHCINITDFGVCHLLRNCHRLQYVDL |
| | ACCQELTNDTLFELSQLPRLRRIGLVKCHNITD |
| | HGILYLANNRRSPDDTLERVHLLYCTQISIFPI |
| | YKLLMACRRLTHLSLTGIRDFLRSDITRFCRDP |
| | PNDFTQSQRDMFCVFSGDGVRKLRDHLSSLYHQ |
| | QQQINRYVNSQNIGNLRDDGETLNEIFQYIANP |
| | ATPGQLPPRVQELVEARRRNRNQERIMTNTVNF |
| | PEQIRRLSLLPPEQQQALPQPIRQLIAQATASP |
| | LSFPLQDQEPQQQQQQERGLGIPPVDNFSPVVD |
| | EGQEYDEDQEME |
| SEQ ID | MQSNSERDSSPSDSNSTIELQRSENEYDHLTNT |
| NO: 10 | IMEDLGQKLNHYKESQDTSSSHILHLPTEVLLL |
| PpGRR1 | ILSFVTSKTDLLSFMLTCRKFGDLVSGLLWFRP |
| (R617C) | GISNAYVYKEMIRIMRIPPEKTFWDYKKFIRRL |
| mutant | NLSLVSNLVEDEFLYAFSGCPNLERITLVNCSK |
| | VTADSVATILKDASNLQSIDLTGVVNITDGVYY |
| | SLARHCKKLQGLYAPGSMAVSKNAVYTLISNCP |
| | MLKRIKLSECVGVDDEIVVKLVRECKNLVELDL |
| | HGCIRVTDYALVVLFEELEYLREFKISMNDHIT |
| | ERCFLGLPNEPYLDKLRIIDFTSCSNVNDKLVI |
| | KLVQLAPKLRHIVLSKCTKITDSSLRALATLGK |
| | CLHYLHLGHCINITDFGVCHLLRNCHRLQYVDL |
| | ACCQELTNDTLFELSQLPRLRRIGLVKCHNITD |
| | HGILYLANNRRSPDDTLERVHLSYCTQISIFPI |
| | YKLLMACRRLTHLSLTGIRDFLRSDITRFCRDP |
| | PNDFTQSQRDMFCVFSGDGVRKLRDHLSSLYHQ |
| | QQQINRYVNSQNIGNLRDDGETLNEIFQYIANP |
| | ATPGQLPPRVQELVEARRRNRNQERIMTNTVNF |
| | PEQIRRLSLLPPEQQQALPQPICQLIAQATASP |
| | LSFPLQDQEPQQQQQQERGLGIPPVDNFSPVVD |
| | EGQEYDEDQEME |
| SEQ ID | MDQDNNNHNDSNRLHPPDIHPNLGPQLWLNSSG |
| NO: 11 | DFDDNNNSTRPQMPSRTRETATSERNASEVRDA |
| ScGRR1 | TLNNIFRFDSIQRETLLPTNNGQPLNQNFSLTF |
| | QPQQQTNALNGIDINTVNTNLMNGVNVQIDQLN |
| | RLLPNLPEEERKQIHEFKLIVGKKIQEFLVVIE |
| | KRRKKILNEIELDNLKLKELRIDNSPQAISYLH |
| | KLQRMRLRALETENMEIRNLRLKILTIIEEYKK |
| | SLYAYCHSKLRGQQVENPTDNFIIWINSIDTTE |
| | SSDLKEGLQDLSRYSRQFINNVLSNPSNQNICT |
| | SVTRRSPVFALNMLPSEILHLILDKLNQKYDIV |
| | KFLTVSKLWAEIIVKILYYRPHINKKSQLDLFL |
| | RTMKLTSEETVFNYRLMIKRLNFSFVGDYMHDT |
| | ELNYFVGCKNLERLTLVFCKHITSVPISAVLRG |
| | CKFLQSVDITGIRDVSDDVFDTLATYCPRVQGF |
| | YVPQARNVTFDSLRNFIVHSPMLKRIKITANNN |
| | MNDELVELLANKCPLLVEVDITLSPNVTDSSLL |
| | KLLTRLVQLREFRITHNTNITDNLFQELSKVVD |
| | DMPSLRLIDLSGCENITDKTIESIVNLAPKLRN |
| | VFLGKCSRITDASLFQLSKLGKNLQTVHFGHCF |
| | NITDNGVRALFHSCTRIQYVDFACCTNLTNRTL |
| | YELADLPKLKRIGLVKCTQMTDEGLLNMVSLRG |
| | RNDTLERVHLSYCSNLTIYPIYELLMSCPRLSH |
| | LSLTAVPSFLRPDITMYCRPAPSDFSENQRQIF |
| | CVFSGKGVHKLRHYLVNLTSPAFGPHVDVNDVL |
| | TKYIRSKNLIFNGETLEDALRRIITDLNQDSAA |
| | IIAATGLNQINGLNNDFLFQNINFERIDEVFSW |
| | YLNTFDGIRMSSEEVNSLLLQVNKTFCEDPFSD |
| | VDDDQDYVVAPGVNREINSEMCHIVRKFHELND |
| | HIDDFEVNVASLVRVQFQFTGFLLHEMTQTYMQ |
| | MIELNRQICLVQKTVQESGNIDYQKGLLIWRLL |
| | FIDKFIMVVQKYKLSTVVLRLYLKDNITLLTRQ |
| | RELLIAHQRSAWNNNNDNDANRNANNIVNIVSD |
| | AGANDTSNNETNNGNDDNETENPNFWRQFGNRM |
| | QISPDQMRNLQMGLRNQNMVRNNNNNTIDESMP |
| | DTAIDSQMDEASGTPDEDML |

### REFERENCES

Barnard G C, Kull A R, Sharkey N S, Shaikh S S, Rittenhour A M, Burnina I, Jiang Y, Li F, Lynaugh H, Mitchell T, Nett J H, Nylen A, Potgieter T I, Prinz B, Rios S E, Zha D, Sethuraman N, Stadheim T A, Bobrowicz P (2010) High-throughput screening and selection of yeast cell lines expressing monoclonal antibodies. J. Ind. Microbiol. Biotechnol. 37(9):961-71.
Bobrowicz P, Davidson R C, Li H, Potgieter T I, Nett J H, Hamilton S R, Stadheim T A, Miele R G, Bobrowicz B, Mitchell T, Rausch S, Renfer E, Wildt S (2004) Engineering of an artificial glycosylation pathway blocked in core oligosaccharide assembly in the yeast Pichia pastoris: production of complex humanized glycoproteins with terminal galactose. Glycobiology 14(9):757-66.
Carter P, Presta L, Gorman C M, Ridgway J B, Henner D, Wong W L, Rowland A M, Kotts C, Carver M E, Shepard H M. Humanization of an anti-p185HER2 antibody for human cancer therapy. Proc Natl Acad Sci USA. 1992 May 15; 89(10):4285-9. PubMed PMID: 1350088
Choi B K, Bobrowicz P, Davidson R C, Hamilton S R, Kung D H, Li H, Miele R G, Nett J H, Wildt S, Gerngross T U (2003) Use of combinatorial genetic libraries to humanize N-linked glycosylation in the yeast Pichia pastoris. Proc Natl Acad Sci USA. 100(9):5022-7.
Hamilton S R, Davidson R C, Sethuraman N, Nett J H, Jiang Y, Rios S, Bobrowicz P, Stadheim T A, Li H, Choi B K, Hopkins D, Wischnewski H, Roser J, Mitchell T, Strawbridge R R, Hoopes J, Wildt S, Gerngross T U (2006) Humanization of yeast to produce complex terminally sialylated glycoproteins. Science 313(5792):1441-3.
Hopkins D, Gomathinayagam S, Rittenhour A M, Du M, Hoyt E, Karaveg K, Mitchell T, Nett J H, Sharkey N J, Stadheim T A, Li H, Hamilton S R. Elimination of {beta}-mannose glycan structures in Pichia pastoris. Glycobiology. 2011 Aug. 12. [Epub ahead of print] PubMed PMID: 21840970.
Jiang Y, Li F, Zha D, Potgieter T I, Mitchell T, Moore R, Cukan M, Houston-Cummings N R, Nylen A, Drummond J E, McKelvey T W, d'Anjou M, Stadheim T A, Sethuraman N, Li H. Purification process development of a recombinant monoclonal antibody expressed in glycoengineered Pichia pastoris. Protein Expr Purif. 2011 March;76(1):7:6-14. Epub 2010 Nov. 11. PubMed PMID: 21074617.
Li H, Sethuraman N, Stadheim T A, Zha D, Prinz B, Ballew N, Bobrowicz P, Choi B K, Cook W J, Cukan M, Houston-Cummings N R, Davidson R, Gong B, Hamilton S R, Hoopes J P, Jiang Y, Kim N, Mansfield R, Nett J H, Rios S, Strawbridge R, Wildt S, Gerngross T U (2006) Optimization of humanized IgGs in glycoengineered Pichia pastoris. Nat Biotechnol. 24(2):210-5.
Pang S, Vinters H V, Akashi T, O'Brien W A, Chen I S. HIV-1 env sequence variation in brain tissue of patients with AIDS-related neurologic disease. J Acquir Immune Defic Syndr. 1991; 4(11):1082-92. PubMed PMID: 1684385.
Potgieter T I, Cukan M, Drummond J E, Houston-Cummings N R, Jiang Y, Li F, Lynaugh H, Mallem M, McKelvey T W, Mitchell T, Nylen A, Rittenhour A, Stadheim T A, Zha D, d'Anjou M. (2009) Production of monoclonal antibodies by glycoengineered Pichia pastoris. J. Biotechnol. 139(4):318-25.
Traven A, Jelicic B, Sopta M. (2006) Yeast GAL4: a transcriptional paradigm revisited. EMBO Rep. 7(5):496-9.
Varadarajan R, Sharma D, Chakraborty K, Patel M, Citron M, Sinha P, Yadav R, Rashid U, Kennedy S, Eckert D, Geleziunas R, Bramhill D, Schleif W, Liang X, Shiver J. Characterization of gp120 and its single-chain derivatives, gp120-CD4D12 and gp120-M9: implications for targeting the C D4i epitope in human immunodeficiency virus vaccine design. J Virol. 2005 February; 79(3):1713-23.
Winston F (2008) EMS and U V Mutagenesis in Yeast. Curr. Protoc. Mol. Biol. 82:13.3B.1-13.3B.5
Wurm F M. Production of recombinant protein therapeutics in cultivated mammalian cells. Nat Biotechnol. 2004 November; 22(11):1393-8. Review. PubMed PMID: 15529164.
Zhang N, Liu L, Dan Dumitru C, Cummings N R, Cukan M, Jiang Y, Li Y, Li F, Mitchell T, Mallem M R, Ou Y, Patel R N, Vo K, Vo K, Wang H, Burnina I, Choi B K, Huber H E, Stadheim T A, Zha D. Glycoengineered Pichia produced anti-HER2 is comparable to trastuzumab in preclinical study. MAbs. 2011 May 1; 3(3). [Epub ahead of print] PubMed PMID: 21487242.

What is claimed is:

1. An engineered lower eukaryotic host cell that has a modified GRR1 gene.

2. The host cell of claim 1, wherein the GRR1 gene has been modified by: (i) reducing or eliminating the expression of a GRR1 gene or polypeptide, or (ii) introducing a mutation in a GRR1 gene.

3. The host cell of claim 1 or 2, further comprising a mutation, disruption or deletion of one or more genes encoding protease activities, alpha-1,6-mannosyltransferase activities, alpha-1,2-mannosyltransferase activities, mannosylphosphate transferase activities, β-mannosyltransferase activities, 0-mannosyltransferase (PMT) activities, and/or dolichol-P-Man dependent alpha(1-3) mannosyltransferase activities.

4. The host cell of any one of claims 1-3, further comprising one or more nucleic acids encoding one or more glycosylation enzymes selected from the group consisting of: glycosidases, mannosidases, phosphomannosidases, phosphatases, nucleotide sugar transporters, nucleotide sugar epimerases, mannosyltransferases, N-acetylglucosaminyltransferases, CMP-sialic acid synthases, N-acetylneuraminate-9-phosphate synthases, galactosyltransferases, sialyltransferases, and oligosaccharyltransferases.

5. The host cell of any one of claims 1-4, further comprising a nucleic acid encoding a recombinant protein.

6. The host cell of claim 5, wherein the recombinant protein is selected from the group consisting of: an antibody (IgA, IgG, IgM or IgE), an antibody fragment, kringle domains of the human plasminogen, erythropoietin, cytokines, coagulation factors, soluble IgE receptor α-chain, urokinase, chymase, urea trypsin inhibitor, IGF-binding protein, epidermal growth factor, growth hormone-releasing factor, annexin V fusion protein, angiostatin, vascular endothelial growth factor-2, myeloid progenitor inhibitory factor-1, osteoprotegerin, α-1 antitrypsin, DNase II, α-feto proteins, insulin, Fc-fusions, and HSA-fusions.

7. The host cell of any one of claims 1-6, wherein the cell exhibits an increase in culture stability, thermal tolerance and/or improved fermentation robustness compared with a GRR1 naïve parental host cell under similar culture conditions.

8. The host cell of claim 7, wherein the cell is capable of surviving in culture at 32° C. for at least 80 hours of fermentation with minimal cell lysis.

9. The host cell of any one of the above claims, wherein the host cell is glyco-engineered.

10. The host cell of any one of the above claims, wherein the host cell lacks OCH1 activity.

11. The host cell of any one of the above claims, wherein the host cell is a fungal host cell.

12. The host cell of any one of the above claims, wherein the host cell is a yeast host cell.

13. The host cell of any one of the above claims, wherein the host cell is a Pichia sp. host cell.

14. The host cell of claim 13, wherein the host cell is Pichia pastoris.

15. The host cell of claim 14, wherein the GRR1 gene encodes a polypeptide comprising the amino acid sequence set forth in SEQ ID NO:6 or a natural variant (polymorph) of said polypeptide.

16. A method for producing a heterologous polypeptide in an engineered lower eukaryotic host cell, said method comprising: (a) introducing a polynucleotide encoding a heterologous polypeptide into the host cell of any one of claims 1-15; (b) culturing said host cell under conditions favorable to the expression of the heterologous polypeptide; and, optionally, (c) isolating the heterologous polypeptide from the host cell.

17. An isolated nucleic acid encoding a wild-type or mutated GRR1 gene or fragment thereof.

18. The isolated nucleic acid of claim 17, wherein an isolated host cell expressing said nucleic acid exhibits an increase in culture stability, thermal tolerance and/or improved fermentation robustness compared to a GRR1 naive parental host cell under similar conditions.

19. The nucleic acid of claim 17 or 18, selected from the group consisting of:
a. a nucleotide sequence encoding SEQ ID NO:6 or a fragment thereof,
b. a nucleotide sequence encoding SEQ ID NO:7 or a fragment thereof,
c. a nucleotide sequence encoding SEQ ID NO:8 or a fragment thereof,
d. a nucleotide sequence encoding SEQ ID NO:9 or a fragment thereof, and
e. a nucleotide sequence encoding SEQ ID NO:10 or a fragment thereof.

20. An isolated vector comprising the nucleic acid of any one of claims 17-19.
